(19) United States
(12) Patent Application Publication
(10) Pub. No.: US 2026/0102521 A1
(43) Pub. Date: Apr. 16, 2026

(54) METHODS AND SYSTEMS FOR IMPROVED NUCLEIC ACID DELIVERY VIA ULTRASOUND

(71) Applicant: SonoThera, Inc., South San Francisco, CA (US)

(72) Inventors: FEINSTEIN B. Steven, Highland Park, IL (US); GREENBERG Kenneth, Berkley, CA (US); CAMPBELL Barry, San Mateo, CA (US); KRIVEGA Ivan, Burlingame, CA (US)

(21) Appl. No.: 19/228,709

(22) Filed: Jun. 4, 2025

Related U.S. Application Data
(63) Continuation of application No. PCT/US2023/082362, filed on Dec. 4, 2023.
(60) Provisional application No. 63/430,338, filed on Dec. 5, 2022.
(60) Provisional application No. 63/483,193, filed on Feb. 3, 2023.

(51) Int. Cl. A61K 48/00; A61K 38/20; A61K 38/45; A61K 41/00; A61M 37/00; C07K 16/28

(52) U.S. Cl. CPC A61K 48/0066; A61K 38/204; A61K 38/45; A61K 41/0047; A61K 48/0033; A61M 37/0092; C07K 16/28; A61M 2037/0007

(57) ABSTRACT

Provided are methods for improving an expression of a nucleic acid construct in a cell or an organ of a subject using sonoporation and optimization of ultrasonic acoustic energy mechanical index.

### CROSS REFERENCE TO RELATED APPLICATIONS

This application is a continuation of PCT/US2023/082362 filed Dec. 4, 2023, which claims priority to and the benefit of U.S. Provisional Patent Application No. 63/430,338, filed Dec. 5, 2022, and U.S. Provisional Patent Application No. 63/483,193, filed Feb. 3, 2023, each of which is incorporated herein by reference in its entirety and for all purposes.

### BACKGROUND

Gene therapy, in which a functional copy of a gene is transfected into a cell, has been proposed as a possible method of treating genetic diseases. However, prior art methods of gene therapy using ultrasound or sonoporation suffer from significant shortcomings such as low transfection rates, and insufficient gene expression, which have prevented the clinical development and commercialization of these methodologies. There remains a need in the art for an effective gene therapy technique that can transfect a gene to a cell in an organ or a tissue in a subject in a safe, effective, and durable manner.

### SUMMARY OF THE INVENTION

Responsive to the unmet need in the art, provided herein are methods for delivering a nucleic acid payload (e.g., an expression cassette comprising a transgene or therapeutic oligonucleotide) to a target cell by sonoporation. In one aspect, the present disclosure provides methods for delivery of the nucleic acid payload to a target cell by optimizing parameters or protocols of applied ultrasonic acoustic energy, including methods for increasing or decreasing expression of a gene in a target cell by applying ultrasonic acoustic energy at alternating mechanical indexes to induce stable vibration cavitation, and inertial cavitation of the sonoactive microstructures. In some cases, the nucleic acid payload is a miniplasmid, and delivery of the alternating mechanical indexes to induce stable vibration cavitation, and inertial cavitation of the sonoactive microstructures enhances delivery of the miniplasmid to the target cell.

Aspects disclosed herein provide a method of delivering a nucleic acid payload to a target cell of a subject comprising: administering to the subject a nucleic acid construct comprising the nucleic acid payload, wherein the nucleic acid construct is a miniplasmid; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the miniplasmid is less than or equal to 500 base pairs in length excluding an expression cassette. In some embodiments, the nucleic acid construct is administered systemically. In some embodiments, applying the ultrasonic acoustic energy at the first MI and applying the ultrasonic acoustic energy at the second MI are repeated at least twice. In some embodiments, applying the ultrasonic acoustic energy at the first MI and applying the ultrasonic acoustic energy at the second MI are repeated from 4 to 18 times. In some embodiments, applying the ultrasonic acoustic energy at the first MI and applying the ultrasonic acoustic energy at the second MI are repeated from 6 to 12 times. In some embodiments, applying the ultrasonic acoustic energy at the first MI and applying the ultrasonic acoustic energy at the second MI are repeated from 8 to 10 times. In some embodiments, applying the ultrasonic acoustic energy at the first MI and applying the ultrasonic acoustic energy at the second MI are repeated 9 times. In some embodiments, an ultrasound transducer is continuously in contact with the subject during applying the ultrasonic acoustic energy at the first MI and applying the ultrasonic acoustic energy at the second MI. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy of d. comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of about 100 μs to about 3300 μs. In some embodiments, applying the ultrasonic acoustic energy at the second MI comprises applying the ultrasonic acoustic energy at the second MI using a pulse with a duration of about 200 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of about 1 μs to about 5 μs. In some embodiments, applying the ultrasonic acoustic energy at the second MI comprises applying the ultrasonic acoustic energy at the second MI using a pulse with a duration of about 2.3 μs. In some embodiments, applying the ultrasonic acoustic energy at the second MI comprises applying the ultrasonic acoustic energy at the second MI using a pulse with a duration of at least 2.3 μs. In some embodiments, applying the ultrasonic acoustic energy at the first MI comprises initially applying the ultrasonic acoustic energy at the first MI from about 2 s to about 30 s. In some embodiments, the method includes repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI. In some embodiments, the ultrasonic acoustic energy of (c) and the ultrasonic acoustic energy of (d) are applied for a total amount of time ranging from about 1 s to about 60 m. In some embodiments, the ultrasonic acoustic energy of (c) and the ultrasonic acoustic energy of (d) are applied for a total amount of time ranging from about 60 s to about 120 s. In some embodiments, the first MI ranges from about 0.05 to about 0.3. In some embodiments, the first MI ranges from about 0.09 to about 0.3. In some embodiments, the second MI ranges from about 1.0 to about 1.8. In some embodiments, the second MI ranges from about 1.4 to about 1.8. In some embodiments, the second MI ranges from about 1.4 to about 2.0. In some embodiments, the nucleic acid construct is a circular nucleic acid. In some embodiments, the nucleic acid construct is a miniplasmid. In some embodiments, the miniplasmid comprises less than 500 base pairs excluding an expression cassette. In some embodiments, the miniplasmid does not comprise antibiotic resistant genes. In some embodiments, the miniplasmid does not comprise a bacterial genome. In some embodiments, the nucleic acid construct enhances the expression of the nonendogenous gene. In some embodiments, the method induces expression of the nucleic acid payload in the target cell within 20 hours of the applying the ultrasonic acoustic energy. In some embodiments, the nucleic acid construct is configured to perform gene augmentation, gene replacement, base editing, base knockdown, gene editing gene knockdown, or gene knockout. In some embodiments, the nucleic acid construct is configured for enhanced stability in vivo. In some embodiments, the nucleic acid construct is administered at a dose of about 100 μg to about 200 ug. In some embodiments, the nucleic acid construct is administered at a dose of about 0.5 mg/kg to about 32 mg/kg. In some embodiments, about 2×10\hat{ }13 to about 3×10\hat{ }13 copies of the nucleic acid construct are administered to the subject. In some embodiments, the miniplasmid comprises a therapeutic transgene and/or a regulatory element. In some embodiments, applying ultrasonic acoustic energy at the first MI induces stable vibration cavitation of the sonoactive microstructures. In some embodiments, applying ultrasonic acoustic energy at the first MI does not induce substantial disruption of the sonoactive microstructures (e.g., bursting or inertial cavitation). In some embodiments, applying ultrasonic acoustic energy at the first MI does not induce substantial disruption of the sonoactive microstructures in a vascular space and an extravascular space, or induces stable vibration cavitation of the sonoactive microstructures in a vascular space and an extravascular space. In some embodiments, applying ultrasonic acoustic energy at the second MI induces inertial cavitation of the sonoactive microstructures to disrupt the sonoactive microstructures. In some embodiments, applying ultrasonic acoustic energy at the second MI induces inertial cavitation of the sonoactive microstructures to disrupt the sonoactive microstructures in a vascular space and an extravascular space. In some embodiments, the extravascular spaces comprise an interstitial space, a subcutaneous space, an intramuscular inter-osseous space, or a lymphatic space. In some embodiments, the extravascular spaces comprise an extravascular tissue. In some embodiments, the extravascular tissue comprises an interstitial space, a cytoplasmic space, a subcutaneous, a lymph tissues, a muscle, or combinations thereof. In some embodiments, the method does not result in substantial cellular damage to the target cell. In some embodiments, the method results in less than 1%, 5%, or 10% of target cells undergoing apoptosis. In some embodiments, the following biomarkers for cellular damage are not detected at apoptotic levels following (a)-(d): ALT, AST, IL6, BCL2, or combinations thereof, and, optionally wherein the target cell is in a liver. In some embodiments, the following biomarkers for cellular damage are not clinically elevated following (a)-(d): ALT, AST, IL6, BCL2, or combinations thereof, and, optionally wherein the target cell is in a liver. In some embodiments. ALT is not detected at levels exceeding 60, 65, 70, 75, 80, 85, 90, 95, 100, 125, 150, 175, or 200 U/L following (a)-(d). In some embodiments. AST is not detected at levels exceeding 225, 250, 275, or 300 U/L following (a)-(d). In some embodiments. IL6 is not detected at levels exceeding 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, or 6 μg/mL following (a)-(d). In some embodiments, the target cell is in a liver. In some embodiments, the target cell is in a kidney. In some embodiments, the target cell is in a heart or skeletal muscle. In some embodiments, the target cell is in a brain. In some embodiments, the target cell is in a pancreas. In some embodiments, the target cell is in a tumor, or is a tumor cell. In some embodiments, applying the ultrasound acoustic energy at the first mechanical index induces formation of an intercellular gap or an interendothelial gap. In some embodiments, the intercellular gap or the interendothelial gap ranges from about 10 nm to about 10 μm. In some embodiments, the method includes moving the nucleic acid construct from an intravenous space into an interstitial space. In some embodiments, the method includes moving the nucleic acid construct from an interstitial space to an intracellular space. In some embodiments, the stable vibration cavitation of the sonoactive microstructures moves the nucleic acid construct from an intravenous space into an interstitial space. In some embodiments, the inertial cavitation further moves the nucleic acid construct from an interstitial space into an intracellular space. In some embodiments, applying the ultrasound acoustic energy at the second mechanical index induces formation of a pore in a membrane of the cell. In some embodiments, the formation of a pore in a membrane of the cell ranges from about 10 nm to about 10 μm. In some embodiments, the nucleic acid payload comprises a transgene. In some embodiments, the transgene comprises a therapeutic transgene. In some embodiments, the transgene comprises a detectible marker. In some embodiments, the transgene comprises luciferase. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising SERP. In some embodiments, the nucleic acid construct comprises a promoter sequence comprising P3. In some embodiments, the method comprises inducing expression of the nucleic acid payload in the target cell. In some embodiments, inducing expression of the nucleic acid payload comprises inducing expression of luciferase. In some embodiments, inducing expression of the nucleic acid payload comprises inducing a flux which is 2, 3, 4, or 5× greater than expression induced without repeating (c) and (d). In some embodiments, inducing expression of the nucleic acid payload comprises inducing a flux of at least 10\hat{ }6 p/s. In some embodiments, inducing expression of the nucleic acid payload comprises inducing a flux of about 10\hat{ }6 p/s to about 10\hat{ }9 p/s. In some embodiments, inducing expression of the nucleic acid payload comprises inducing production of RNA encoded by the payload. In some embodiments, inducing expression of the nucleic acid payload comprises inducing production of protein encoded by the payload. In some embodiments, the sonoactive microstructures are administered at a concentration of about 5×10\hat{ }8 to about 1.2×10\hat{ }9 microstructures/mL. In some embodiments, the sonoactive microstructures comprise sonazoid microbubbles. In some embodiments, the sonoactive microstructures comprise a lipid stabilized microstructure. In some embodiments, the sonoactive microstructures comprise a phospholipid stabilized microstructure. In some embodiments, the phospholipid stabilized microstructure comprises a high molecular weight gas core, or a perflutran core. In some embodiments, the sonoactive microstructures are administered at a concentration of about 10\hat{ }9 microstructures/mL. In some embodiments, the sonoactive microstructures are administered at a concentration of about 0.1 to about 0.8 mL/kg. In some embodiments, the sonoactive microstructures are administered at a concentration of about 0.1 to about 20.0 mL/kg. In some embodiments, the sonoactive microstructures comprise a protein stabilized microstructure. In some embodiments, the sonoactive microstructures comprise optison microbubbles. In some embodiments, the sonoactive microstructures are administered at a concentration of about 5×10\hat{ }8 to about 8×10\hat{ }8 microstructures/mL. In some embodiments, the ultrasound acoustic energy is applied at a distance of about 0.5 cm to about 20 cm from the target cell. In some embodiments, the nucleic acid construct and the sonoactive microstructures are coadministered. The method of any of the immediately preceding claim, wherein the nucleic acid construct and the sonoactive microstructures are mixed prior to being coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occurs sequentially. In some embodiments, the administering of the nucleic acid construct and the sonoactive microstructures occurs continuously. In some embodiments, the administering of the nucleic acid construct and the sonoactive microstructures is by intravenous administration. In some embodiments, the administering of the nucleic acid construct and the sonoactive microstructures intramuscular, subcutaneous, inter-osseous or retrovesiclar administration. In some embodiments, inducing expression of the nucleic acid payload comprises inducing expression within about 3 hours of administering the payload. In some embodiments, inducing expression of the nucleic acid payload comprises inducing expression within about 6 hours of administering the payload. In some embodiments, inducing expression of the nucleic acid payload comprises inducing expression within about 12 hours of administering the payload. In some embodiments, inducing expression of the nucleic acid payload comprises inducing expression in a cell in a liver. In some embodiments, inducing expression of the nucleic acid payload comprises inducing expression in a cell in a kidney. In some embodiments, the method includes inducing expression of the nucleic acid payload and maintaining expression of a protein encoded by the nucleic acid payload for at least 1, 2, 3, 4, 5, 6, or 7 days. In some embodiments, the method includes inducing expression of the nucleic acid payload and maintaining expression of a protein encoded by the nucleic acid payload for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 days. In some embodiments, the method increases durability of expression of a protein encoded by the nucleic acid payload. In some embodiments, the method includes increasing expression of the nucleic acid payload by increasing the dosage of the nucleic acid payload administered to the subject. In some embodiments, the method includes increasing expression of the nucleic acid payload by increasing the dosage of the nucleic acid payload administered to the subject in a linear manner. In some embodiments, the method includes increasing expression of the nucleic acid payload by administering at least 5, 50, 250, or 500 μg of the nucleic acid payload to the subject. In some embodiments, delivering the nucleic acid payload to the target cell of the subject increases or decreases expression of a gene in the target cell. In some embodiments, the nucleic acid payload is delivered to the target cell, resulting in a copy number of the nucleic acid payload per diploid genome of at least 0.15. In some embodiments, the nucleic acid payload is delivered to the target cell, resulting in a copy number of the nucleic acid payload per diploid genome of at least 0.2. In some embodiments, the nucleic acid payload is delivered to the target cell, resulting in a copy number of the nucleic acid payload per diploid genome of 0.15 to 0.3. In some embodiments, the therapeutic transgene comprises a nucleic acid sequence encoding FVIII. In some embodiments, the therapeutic transgene comprises a nucleic acid sequence encoding FIX. In some embodiments, the therapeutic transgene comprises a nucleic acid sequence encoding COL4A3. In some embodiments, the therapeutic transgene comprises a nucleic acid sequence encoding COL4A4. In some embodiments, the therapeutic transgene comprises a nucleic acid sequence encoding COL4A5. In some embodiments, the therapeutic transgene comprises a nucleic acid sequence encoding PKD1. In some embodiments, the therapeutic transgene comprises a nucleic acid sequence encoding PKD2. In some embodiments, delivering the nucleic acid payload to the target cell of the subject increases or decreases expression of a gene in the target cell.

Aspects disclosed herein provide a kit comprising: a first container comprising microbubbles for sonoporation; and a second container comprising miniplasmids comprising a transgene. In some embodiments, the miniplasmid further comprises an expression cassette. In some embodiments, the first container and second container are configured to induce the expression of the transgene in the target cell of the subject within 20 hours after the transfection. In some embodiments, the kit further includes instructions for operation of an ultrasound machine hardware and software parameters sufficient to disrupt the sonoactive microstructures. In some embodiments, the kit further includes comprising instructions for administration of the first container and the second container.

Aspects disclosed herein provide a system comprising: an ultrasound transducer configured to apply ultrasound acoustic energy to a subject at a plurality of mechanical indexes; a computer system comprising a computer processor and a computer-readable medium, wherein the computer system is configured to implement a method of applying ultrasonic acoustic energy to a target cell of the subject, the method comprising: applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0), wherein the subject has been administered a nucleic acid construct comprising the nucleic acid payload, wherein the nucleic acid construct is a miniplasmid, and a plurality of sonoactive microstructures. In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the nucleic acid construct is a plasmid that is less than or equal to 500 base pairs in length excluding an expression cassette, or wherein the wherein the nucleic acid construct is a miniplasmid. In some embodiments, applying the ultrasonic acoustic energy at the first MI and applying the ultrasonic acoustic energy at the second MI are repeated at least twice. In some embodiments, applying the ultrasonic acoustic energy at the first MI and applying the ultrasonic acoustic energy at the second MI are repeated from 4 to 18 times. In some embodiments, applying the ultrasonic acoustic energy at the first MI and applying the ultrasonic acoustic energy at the second MI are repeated from 6 to 12 times. In some embodiments, applying the ultrasonic acoustic energy at the first MI and applying the ultrasonic acoustic energy at the second MI are repeated from 8 to 10 times. In some embodiments, the second MI ranges from about 1.4 to about 2.0. In some embodiments, applying the ultrasound acoustic energy at the second mechanical index induces formation of a pore in a membrane of the cell. In some embodiments, applying the ultrasound acoustic energy at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy of d. comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of about 2.3 μs. In some embodiments, the method includes repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI. Aspects disclosed herein provide a computer-readable medium configured to implement a method of applying ultrasonic acoustic energy to a target cell of the subject, the method comprising: applying an ultrasonic acoustic energy to the target cell at a first mechanical index (MI) that is up to 0.4; and applying an ultrasonic acoustic energy to the target cell at a second MI that is greater than 0.4 and up to 2.0, wherein the subject has been administered (1) a nucleic acid construct comprising a nucleic acid payload, wherein the nucleic acid construct is a miniplasmid, and (2) a plurality of sonoactive microstructures. The computer readable medium of claim 153, wherein an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the miniplasmid is less than or equal to 500 base pairs in length excluding an expression cassette. In some embodiments, applying the ultrasonic acoustic energy at the first MI and applying the ultrasonic acoustic energy at the second MI are repeated at least twice. In some embodiments, applying the ultrasonic acoustic energy at the first MI and applying the ultrasonic acoustic energy at the second MI are repeated from 4 to 18 times. In some embodiments, applying the ultrasonic acoustic energy at the first MI and applying the ultrasonic acoustic energy at the second MI are repeated from 6 to 12 times. In some embodiments, applying the ultrasonic acoustic energy at the first MI and applying the ultrasonic acoustic energy at the second MI are repeated from 8 to 10 times. In some embodiments, the second MI ranges from about 1.4 to about 2.0. In some embodiments, applying the ultrasound acoustic energy at the second mechanical index induces formation of a pore in a membrane of the cell. In some embodiments, applying the ultrasound acoustic energy at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy at the second MI comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of about 2.3 μs. In some embodiments, the instructions comprise repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, wherein the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D show vector maps of exemplary nucleic acid constructs used in the present disclosure.

FIG. 2 illustrates results of nucleic acid transfection and expression from IVIS average radiance measurements as compared to a control, in which fluorescence resulting from gene transfection and expression is observed in murine livers.

FIGS. 3A and 3B illustrate results of nucleic acid transfection and expression from IVIS average radiance measurements as compared to a control, in which fluorescence resulting from gene transfection and expression is observed in murine livers.

FIG. 4 illustrates results of nucleic acid transfection and expression from IVIS average radiance measurements using different high MI pulse bursts, in which fluorescence resulting from gene transfection and expression is observed in murine livers.

FIGS. 5A-5C illustrates results of nucleic acid transfection and expression from IVIS average radiance measurements using different high MI pulse bursts, in which fluorescence resulting from gene transfection and expression is observed in murine livers.

FIGS. 6A-6C illustrates results of nucleic acid transfection and expression from IVIS average radiance measurements using different 5 μg, 50 μg, 250 μg, or 500 μg doses of a luciferase nanoplasmid, in which fluorescence resulting from gene transfection and expression is observed in murine livers.

FIG. 7 illustrates results of nucleic acid transfection and expression from IVIS average radiance measurements at 3, 6, 12, 18, 24, and 30 after delivery of a luciferase miniplasmid by sonoporation in four different subject.

FIG. 8A-8C illustrates biomarker levels in mice transfected using sonoporation.

FIG. 9 illustrates the weight of mice following transfection. Mice were weighed daily for one week following transfection.

FIG. 10 illustrates the average radiance of the fluorescent reporter for the 500 ug dose cohort over 7 days.

FIG. 11 illustrates exogenous gene expression levels in liver cells following delivery via sonoporation of a nucleic acid payload by different vectors measured using quantitative polymerase chain reaction (qPCR).

FIG. 12 illustrates an exemplary ultrasound transducer system having computer processors with a computer readable medium storing instructions for implementing the methods of the present disclosure.

### DETAILED DESCRIPTION OF THE INVENTION

Provided herein are methods for nucleic acid transfection into and expression in a cell, tissue, or organ of a subject in a targeted manner using sonoporation (e.g., a process comprising applying an ultrasonic acoustic energy to a cell, tissue, or organ, such as to provide increased porosity in the cell, tissue, or organ). In one aspect, the present disclosure provides methods for delivery of the nucleic acid payload to a target cell by optimizing parameters or protocols of applied ultrasonic acoustic energy, including methods for increasing or decreasing expression of a gene in a target cell by applying ultrasonic acoustic energy at alternating mechanical indexes to induce stable vibration cavitation, and inertial cavitation of the sonoactive microstructures. In some cases, the nucleic acid payload is a miniplasmid, and delivery of the alternating mechanical indexes to induce stable vibration cavitation, and inertial cavitation of the sonoactive microstructures enhances delivery of the miniplasmid to the target cell.

Provided in certain embodiments herein are methods for transfecting a nucleic acid construct into a target cell or tissue (e.g., of a subject) by applying a first ultrasonic acoustic energy to a cell, tissue, or organ, and applying a second ultrasonic acoustic energy to the cell, tissue, or organ. In specific embodiments herein are methods for transfecting a nucleic acid construct into a target cell or tissue by applying a first ultrasonic acoustic energy having a first mechanical index (MI) and applying a second ultrasonic acoustic energy having a second mechanical index (MI). The present disclosure provides methods for enhancing transfection of a nucleic acid construct into the target cell or tissue by applying alternating ultrasonic acoustic energy, the alternating acoustic energy alternating between a first mechanical index (MI) and a second MI. Application of ultrasonic acoustic energy can be repeated several times during sonoporation, such as to increase the efficiency of nucleic acid construct transfection and/or delivery.

In some embodiments, a process provided herein provides sonoporation at two or more different ultrasonic acoustic energies (e.g., a first and second ultrasonic acoustic energy having a first and second MI, respectively). In certain embodiments, a process provided herein provides a process wherein an ultrasonic acoustic energy is continuously applied (e.g., ultrasonic acoustic energy transitions from the first ultrasonic acoustic energy to the second ultrasonic acoustic energy, without a period of no ultrasonic acoustic energy being applied). In certain embodiments, a transitory (e.g., third, fourth, etc.) ultrasonic acoustic energy is applied between application of the first and second ultrasonic acoustic energies.

In some embodiments, a sonoporation treatment (e.g., application of a first ultrasonic acoustic energy, a second ultrasonic acoustic energy, a single cycle of a first ultrasonic acoustic energy and a second ultrasonic acoustic energy, or series of cycles comprising a plurality of applications of a first ultrasonic acoustic energy and a plurality of applications of a second acoustic energy) can last for a few seconds (e.g., 1-100 seconds) or more, such as up to a few minutes (e.g., 1-3 minutes). In specific embodiments, a sonoporation treatment last for 1-30 seconds. In some specific embodiments, a sonoporation treatment lasts for 5-100 seconds. In certain embodiments, a sonoporation treatment lasts for at least 1 minute (e.g., 1-30 minutes).

In some embodiments, a first MI is a Low MI (e.g., less than 0.4). In certain embodiments, a second MI is a High MI (e.g., 0.4 or greater). In some embodiments, a first MI is a Low MI (e.g., less than 0.4) and a second MI is a High MI (e.g., 0.4 or greater). In some embodiments, a second MI is a Low MI (e.g., less than 0.4). In certain embodiments, a first MI is a High MI (e.g., 0.4 or greater). In specific embodiments, a second MI is a Low MI (e.g., less than 0.4) and a first MI is a High MI (e.g., 0.4 or greater).

In some embodiments, a Low MI is <0.3. In specific embodiments, a Low MI is <0.2. In more specific embodiments, a Low MI is <0.1. In still more specific embodiments, a Low MI is about 0.09. In still more specific embodiments, a Low MI is about 0.04. In still more specific embodiments, a Low MI is about 0.03.

In some embodiments, a High MI is ≥0.5. In specific embodiments, a High MI is 0.5 to 2.0 or is between 0.5 and 2.0. In more specific embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is greater than 0.4. In some embodiments, a High MI is ≥0.5. In more specific embodiments, a High MI is 0.5 to 1 or is between 0.5 and 2.0. In some embodiments, a High MI is 1.5. In some embodiments, a High MI is 1.8. In some embodiments, a High MI is 2.0.

In certain embodiments, any process provided herein (e.g., a sonoporation treatment) comprises administering of a continuous ultrasonic acoustic energy (which may have varying energy levels) that alternates (e.g., in identical, similar, or variable periods) between Low MI and High MI. In some embodiments, a low MI (e.g., ≤0.1) (e.g., first) ultrasonic acoustic energy (also referred to herein as a Low MI) is administered to the subject, and a set number pulses (e.g., of less than 30 seconds) of High MI (e.g., second) ultrasonic acoustic energy (also referred to herein as a High MI) is administered to the subject. In some embodiments, a process provided herein comprises administration of a plurality of pulses of high MI (e.g., second) ultrasonic acoustic energy, e.g., during an otherwise continuous administration of a low MI (e.g., first) ultrasonic acoustic energy. In specific embodiments, the number of High MI pulses is about 4 or more, such as up to about 12, or an unlimited number of pulses. In specific embodiments the number of High MI pulses is 6-30. In still more specific embodiments, the number of High MI pulses is between 8, 9, 12, 15, or 18, or any number therebetween. In some embodiments, at least 8, 9, 12, 15, or 18 high MI pulses are administered to the subject in between applications of low MI ultrasound acoustic energy.

In some embodiments, high MI ultrasound acoustic energy is administered in a pulse. In specific embodiments, a pulse length is any suitable length, such as less than 30 seconds. In more specific embodiments, a pulse length is less than 15 seconds. In still more specific embodiments, a pulse length is less than 10 seconds. In yet more specific embodiments, a pulse length is less than 5 seconds. In more specific embodiments, a pulse length is less than 2 seconds. In still more specific embodiments, a pulse length is less than 1 second and/or may be greater than or equal to 1 microsecond. In some embodiments, a pulse length ranges from 100 to 300 microseconds. In some embodiments, a pulse length is up to about 200 microseconds. In some embodiments, a pulse length is up to about 500 microseconds. In some embodiments, a pulse length ranges from 1 to 500 microseconds.

In various embodiments, a High MI ultrasonic acoustic energy is provided first temporally (e.g., first in order). In other embodiments, a Low MI ultrasonic acoustic energy is provided second temporally (e.g., second in order).

In some embodiments, any process provided herein further comprises administering (e.g., systemically administering, such as via infusion) a nucleic acid (e.g., any nucleic acid provided herein) to a subject (e.g., to whom the ultrasonic acoustic energies are applied).

In some embodiments, any process provided herein further comprises administering (e.g., systemically administering, such as via infusion) a sonoactive structure (e.g., any sonoactive structure or microbubble described herein) to a subject (e.g., to whom the ultrasonic acoustic energies are applied).

In certain embodiments, provided herein is a method of delivering a nucleic acid payload in a target cell (e.g., of a tissue or organ) of a subject, the method comprising: (a) administering to the subject a nucleic acid construct comprising the nucleic acid payload; (b) administering to the subject a plurality of sonoactive microstructures; and (c) administering a sonoporation treatment.

In some embodiments, the sonoporation treatment comprises applying an ultrasonic acoustic energy to the target cell (e.g., of a tissue or organ of the subject) (e.g., the ultrasonic acoustic energy having a mechanical index (MI)). In some embodiments, applying an ultrasonic acoustic energy to the target cell comprises applying a first ultrasonic acoustic energy to the target cell and applying a second ultrasonic acoustic energy to the target cell. In some embodiments, the (e.g., first or second) ultrasonic acoustic energy has a first mechanical index (MI). In certain embodiments, (e.g., the other of the first or second) ultrasonic energy has a second mechanical index (MI). In some embodiments, the (e.g., first or second) MI is less than 0.4. In certain embodiments (e.g., the other of the first or second) MI is greater than 0.4 (e.g., and less than 2.0).

In specific embodiments, a first ultrasonic acoustic energy and a second ultrasonic acoustic energy are applied sequentially in a repeated manner.

In certain embodiments, the first (either High MI or Low MI) ultrasonic acoustic energy is applied before or after administration of any other agent, such as the nucleic acid and/or sonoactive structure. In some embodiments, the first ultrasonic acoustic energy is applied after administration of the sonoactive structure to the subject. In certain embodiments, the first ultrasonic acoustic energy is applied after administration of the nucleic acid to the subject. In some embodiments, the first ultrasonic acoustic energy is applied after administration of both the nucleic acid and the sonoactive structure(s).

In some embodiments, the first ultrasonic acoustic energy is administered within 60 minutes of administration of the nucleic acid and/or sonoactive structure(s). In specific embodiments, the first ultrasonic acoustic energy is administered within 30 minutes of administration of the nucleic acid and/or sonoactive structure(s). In more specific embodiments, the first ultrasonic acoustic energy is administered within 5 minutes of administration of the nucleic acid and/or sonoactive structure(s). In still more specific embodiments, the first ultrasonic acoustic energy is administered within 2 minutes of administration of the nucleic acid and/or sonoactive structure(s). In still more specific embodiments, the first ultrasonic acoustic energy may be applied simultaneously with administration of the nucleic acid and/or sonoactive structure(s).

In specific embodiments, the first (e.g., High MI) ultrasonic acoustic energy is applied immediately upon administration (e.g., infusion) or a period of time after administration (e.g., infusion) of the sonoactive structure(s) and/or nucleic acid.

In some embodiments, either the first or second ultrasonic acoustic energy is an ultrasonic acoustic energy (e.g., Low MI) that when applied to a cell, tissue, or organ of a subject results in stable cavitation (or stable vibrational cavitation) of the sonoactive structure and/or a change in the average diameter of the sonoactive structure(s), for example, due to inherent resonance properties of the microbubbles.

In certain embodiments, the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In certain embodiments, either the first or second ultrasonic acoustic energy is an ultrasonic acoustic energy (e.g., Low MI) that when applied to a cell, tissue, or organ of a subject results in stable cavitation (or stable vibrational cavitation) and/or a change in the average diameter of the sonoactive structure(s), and the other of the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In some instances, disruption of cell membrane allows target cells to become permeable to circulating agents such as nucleic acid constructs. In certain instances, such circulating agents can then enter the target cells, tissues or organs, such as in a more rapid manner (e.g., relative to either Low MI or High MI ultrasonic acoustic energy application alone, or in the absence of ultrasonic acoustic energy application).

In some embodiments, the methods herein comprise alternating the ultrasonic acoustic energy applied between a first ultrasonic acoustic energy having a first MI and a second ultrasonic acoustic energy having a second MI. In some embodiments, applying alternating ultrasonic acoustic energy administered to a subject between a first MI and a second MI is performed repeatedly over a number of times, such as to enhance gene transfection into the target cells, tissue or organ (e.g., relative to a similar process wherein a first and second ultrasonic acoustic energy are not used and/or are not alternately applied and/or are not alternately applied repeatedly).

In some embodiments, the applying the ultrasonic acoustic energy comprises applying the ultrasonic acoustic energy at a frequency of about 1 MHz to about 10 MHz. In some embodiments, the applying the ultrasonic acoustic energy comprises applying the ultrasonic acoustic energy at a frequency of about 1 MHz to about 1.1 MHz, about 1 MHz to about 1.5 MHz, about 1 MHz to about 2 MHz, about 1 MHz to about 3 MHz, about 1 MHz to about 4 MHz, about 1 MHz to about 5 MHz, about 1 MHz to about 7 MHz, about 1 MHz to about 8 MHz, about 1 MHz to about 9 MHz, about 1 MHz to about 9.3 MHz, about 1 MHz to about 10 MHz, about 1.1 MHz to about 1.5 MHz, about 1.1 MHz to about 2 MHz, about 1.1 MHz to about 3 MHz, about 1.1 MHz to about 4 MHz, about 1.1 MHz to about 5 MHz, about 1.1 MHz to about 7 MHz, about 1.1 MHz to about 8 MHz, about 1.1 MHz to about 9 MHz, about 1.1 MHz to about 9.3 MHz, about 1.1 MHz to about 10 MHz, about 1.5 MHz to about 2 MHz, about 1.5 MHz to about 3 MHz, about 1.5 MHz to about 4 MHz, about 1.5 MHz to about 5 MHz, about 1.5 MHz to about 7 MHz, about 1.5 MHz to about 8 MHz, about 1.5 MHz to about 9 MHz, about 1.5 MHz to about 9.3 MHz, about 1.5 MHz to about 10 MHz, about 2 MHz to about 3 MHz, about 2 MHz to about 4 MHz, about 2 MHz to about 5 MHz, about 2 MHz to about 7 MHz, about 2 MHz to about 8 MHz, about 2 MHz to about 9 MHz, about 2 MHz to about 9.3 MHz, about 2 MHz to about 10 MHz, about 3 MHz to about 4 MHz, about 3 MHz to about 5 MHz, about 3 MHz to about 7 MHz, about 3 MHz to about 8 MHz, about 3 MHz to about 9 MHz, about 3 MHz to about 9.3 MHz, about 3 MHz to about 10 MHz, about 4 MHz to about 5 MHz, about 4 MHz to about 7 MHz, about 4 MHz to about 8 MHz, about 4 MHz to about 9 MHz, about 4 MHz to about 9.3 MHz, about 4 MHz to about 10 MHz, about 5 MHz to about 7 MHz, about 5 MHz to about 8 MHz, about 5 MHz to about 9 MHz, about 5 MHz to about 9.3 MHz, about 5 MHz to about 10 MHz, about 7 MHz to about 8 MHz, about 7 MHz to about 9 MHz, about 7 MHz to about 9.3 MHz, about 7 MHz to about 10 MHz, about 8 MHz to about 9 MHz, about 8 MHz to about 9.3 MHz, about 8 MHz to about 10 MHz, about 9 MHz to about 9.3 MHz, about 9 MHz to about 10 MHz, or about 9.3 MHz to about 10 MHz, including increments therein. In some embodiments, the applying the ultrasonic acoustic energy comprises applying the ultrasonic acoustic energy at a frequency of about 1 MHz, about 1.1 MHz, about 1.5 MHz, about 2 MHz, about 3 MHz, about 4 MHz, about 5 MHz, about 7 MHz, about 8 MHz, about 9 MHz, about 9.3 MHz, or about 10 MHz. In some embodiments, the applying the ultrasonic acoustic energy comprises applying the ultrasonic acoustic energy at a frequency of at least about 1 MHz, at least about 1.1 MHz, at least about 1.5 MHz, at least about 2 MHz, at least about 3 MHz, at least about 4 MHz, at least about 5 MHz, at least about 7 MHz, at least about 8 MHz, at least about 9 MHz, or at least about 9.3 MHz. In some embodiments, the applying the ultrasonic acoustic energy comprises applying the ultrasonic acoustic energy at a frequency of at most at most about 1.1 MHz, at most about 1.5 MHz, at most about 2 MHz, at most about 3 MHz, at most about 4 MHz, at most about 5 MHz, at most about 7 MHz, at most about 8 MHz, at most about 9 MHz, at most about 9.3 MHz, or at most about 10 MHz. In some embodiments, the first ultrasound acoustic energy and the second ultrasound acoustic energy are applied at a same frequency. In some embodiments, the first ultrasound acoustic energy applied at the first MI and the second ultrasound acoustic energy applied at the second MI are applied at a same frequency. In some embodiments, the first ultrasound acoustic energy and the second ultrasound acoustic energy are applied at a different frequency. In some embodiments, the first ultrasound acoustic energy applied at the first MI and the second ultrasound acoustic energy applied at the second MI are applied at a different frequency.

In some embodiments, the method comprises administering ultrasound energy transcutaneously to the subject in proximity to one or more target cells. In some embodiments, the one or more target cells are hepatic cells. In some embodiments, the one or more target cells are renal cells. In some embodiments, the one or more target cells are pancreatic cells. In some embodiments, the one or more target cells are cardiac cells. In some embodiments, the one or more target cells are myocytes. In some embodiments, the one or more target cells are neuronal cells. In some embodiments, the one or more target cells are brain cells. In some embodiments, the one or more target cells are blood cells (e.g., white blood cells). In some embodiments, the target cells are cancerous cells.

In some embodiments, the one or more target cells are comprised in a tissue. In some embodiments, the tissue is skeletal muscle tissue. In some embodiments, the tissue is smooth muscle tissue. In some embodiments, the tissue is connective tissue. In some embodiments, the tissue is lymphatic tissue. In some embodiments, the tissue is nervous tissue. In some embodiments, the tissue is diseased tissue, e.g., cancerous tissue, fibrotic tissue, or tissue otherwise in need of gene therapy.

In some embodiments, the target tissue is comprised in an organ. In some embodiments, the organ is the liver. In some embodiments, the organ is a kidney. In some embodiments, the organ is the pancreas. In some embodiments, the organ is the heart. In some embodiments, the organ is the brain. In some embodiments, the one or more target cells are comprised in a tumor. In some embodiments, the tumor is a solid tumor. In some embodiments, the tumor is a liquid tumor.

In some embodiments, cells, tissue or organ are those of the liver. In some embodiments, cells, tissue or organ are those of the kidney.

In certain embodiments, a subject herein is a mammal. In some embodiments, the mammal is, by way of non-limiting example, a human, rat, mouse, monkey, and other non-human primates.

In certain embodiments, changing parameters of the ultrasound acoustic energy or MI can be performed to induce and/or enhance an expression of a transgene in a cell or an organ of a subject. In one aspect, provided herein are methods of transfection by alternating the ultrasonic acoustic energy using a first MI and a second MI. In some embodiments, the first MI that results in stable vibrational cavitation is applied prior to the second MI, which results in inertial cavitation. In some embodiments, the ultrasonic acoustic energy using the first MI and the second MI are reapplied for a number of times to increase transfection efficiency at the target cell. In some embodiments, during the application of sonoporation, the ultrasonic acoustic energy is applied at the first MI continuously except for when the ultrasonic acoustic energy is applied at the second MI. For example, applying an ultrasonic acoustic energy to the target cell at the first MI then applying an ultrasonic acoustic energy to the target cell at the second MI are repeated between 4 to 18 times. In some embodiments, applying an ultrasonic acoustic energy to the target cell at the first MI then applying an ultrasonic acoustic energy to the target cell at the second MI are repeated an unlimited number of times. In one aspect, during this time, the ultrasonic acoustic energy of the first MI is applied continuously except for when the ultrasonic acoustic energy of the second MI is applied.

In some embodiments, the first MI ranges from about 0.05 to about 0.4. In some embodiments, the first MI ranges from about 0.05 to about 0.3. In some embodiments, the first MI ranges from about 0.05 to about 0.4. In some embodiments, the first MI ranges from about 0.09 to about 0.3.

In some embodiments, the second MI ranges from about 0.5 to about 2.0. In some embodiments, the second MI ranges from greater than 1.4 to about 1.8. In some embodiments, the second MI ranges from greater than 1.4 to about 2.0. In some embodiments, the second MI ranges from about 1.5 to about 2.0.

In some embodiments, applying the ultrasonic acoustic energy at the first MI, and the applying the ultrasonic acoustic energy at the second MI are repeated between 4 and 18 times. In some embodiments, applying the ultrasonic acoustic energy at the first MI, and the applying the ultrasonic acoustic energy at the second MI are repeated between 6 and 12 times. In some embodiments, applying the ultrasonic acoustic energy at the first MI, and the applying the ultrasonic acoustic energy at the second MI are repeated between 8 and 10 times. In some embodiments, applying the ultrasonic acoustic energy at the first MI, and the applying the ultrasonic acoustic energy at the second MI are repeated between 8 and 18 times. In some embodiments, applying the ultrasonic acoustic energy at the first MI, and the applying the ultrasonic acoustic energy at the second MI are repeated 9 times.

In some embodiments, the applying the ultrasound acoustic energy comprises applying the ultrasound acoustic energy of c. or d., without ceasing applying the ultrasound acoustic energy of c. or d. In some embodiments, the applying the ultrasound acoustic energy comprises the ultrasound acoustic energy of c. being applied except for when the ultrasound acoustic energy of d. is applied. In some embodiments, an ultrasound probe applying the ultrasonic acoustic energy is in constant contact with the surface of the subject's skin at the location of application (e.g., abdomen, chest wall, skull, etc.). In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In certain embodiments, a transitory (e.g., third, fourth, etc.) ultrasonic acoustic energy is applied between application of the first and second ultrasonic acoustic energies. In certain embodiments, applying the ultrasound acoustic energy comprises applying the ultrasonic acoustic energy without regard to an EKG gating signal regulating the application of the ultrasound acoustic energy. In certain embodiments, applying the ultrasound acoustic energy comprises applying the ultrasonic acoustic energy without turning off power to the ultrasound transducer off. In some embodiments, applying the ultrasound acoustic energy comprises an ultrasound transducer sending ultrasound acoustic energy or receiving reflected ultrasound acoustic energy at least 95% of a period of time in which an ultrasound transducer continuously is contacting the subject.

In some instances, the ultrasonic acoustic energy of the second MI (e.g., high MI) is applied using a pulse. In some instances, a pulse comprises applying the ultrasonic acoustic energy in a short pulse (e.g., microsecond length pulse). In some cases, the high MI is applied with the pulse, results in induces inertial cavitation and destruction of the sonoactive microstructure, resulting in the disruption of cell membrane and vascular endothelial integrity, transducing the nucleic acid payload to the cell. In some instances, the pulse is applied with a duration of about 1 μs to about 200 μs. In some instances, the pulse is applied with a duration of about 1 μs to about 200 μs or greater.

In some embodiments, (d) comprises applying the ultrasonic acoustic energy at the second MI using a pulse. In some instances, the duration of the second MI applied ranges from 0.1 μs to about 200 μs. In some instances, the duration of the second MI applied ranges from 1 μs to about 200 μs or greater. In some embodiments, (d) comprises applying the ultrasonic acoustic energy at the second MI using a pulse with a duration of about 1 μs to about 200 μs. In some embodiments, (d) comprises applying the ultrasonic acoustic energy at the second MI using a pulse with a duration of up to 200 μs. In some embodiments, (d) comprises applying the ultrasonic acoustic energy at the second MI using a pulse with a duration of about 1 μs to about 500 μs. In some embodiments, (d) comprises applying the ultrasonic acoustic energy at the second MI using a pulse with a duration of up to 500 μs. In some embodiments, (d) comprises applying the ultrasonic acoustic energy at the second MI using a pulse with a duration of about 2.3 μs. In some embodiments, (d) comprises applying the ultrasonic acoustic energy at the second MI using a pulse with a duration of at least 2.3 μs. In some embodiments, (d) comprises applying the ultrasonic acoustic energy at the second MI using a pulse with a duration ranging from 1-500 μs. In some embodiments, (d) comprises applying the ultrasonic acoustic energy at the second MI using a pulse with a duration ranging from 0.1-500 μs.

In some cases, alternating the ultrasonic acoustic energy between the first MI and the second MI for a number of times also allows reperfusion of the sonoactive microstructures and the nucleic acid constructs to the target cell, tissue, or organ, following disruption of the sonoactive microstructures within or proximal to the target cell, tissue, or organ.

In some embodiments, the repeating application of ultrasonic acoustic energy between the first MI and the second MI comprises applying the ultrasound acoustic energy of (c) for an amount of time sufficient to permit reperfusion of the sonoactive microstructures in a tissue comprising the target cell before reapplying the ultrasound acoustic energy of (d).

In some embodiments, the method comprises applying the ultrasound acoustic energy of (c) for 1-30 seconds before repeating the applying the ultrasound acoustic energy of (d). In some embodiments, the method comprises applying the ultrasound acoustic energy of (c) for 5-15 seconds before repeating the applying the ultrasound acoustic energy of (d). In some embodiments, the method comprises applying the ultrasound acoustic energy of (c) for 10 seconds before repeating the applying the ultrasound acoustic energy of (d).

In some instances, the duration of the first MI applied ranges from about 2 s to about 30 s. In some embodiments, (c) comprises initially applying the ultrasonic acoustic energy at the first MI from about 2 s to about 30 s.

In some embodiments, applying the ultrasonic acoustic energy at the first MI, and the applying the ultrasonic acoustic energy at the second MI are repeated for a total amount of time ranging from about 1 s to about 60 m. In some embodiments, applying the ultrasonic acoustic energy at the first MI, and the applying the ultrasonic acoustic energy at the second MI are repeated for a total amount of time ranging from about 60 s to about 120 s.

In some embodiments, applying ultrasonic acoustic energy in (c) induces stable vibration cavitation of the sonoactive microstructures. In some embodiments, applying ultrasonic acoustic energy in (c) does not induce substantial disruption of the sonoactive microstructures. In some embodiments, applying ultrasonic acoustic energy in (c) does not induce substantial disruption of the sonoactive microstructures in a vasculature space and an extravascular space, or induces stable vibration cavitation of the sonoactive microstructures in a vasculature space and an extravascular space.

In some embodiments, (c) induces formation of an intercellular gap or an interendothelial gap or endocytosis. In some embodiments, the intercellular gap or the interendothelial gap ranges from about 10 nm to about 10 μm. In some embodiments, the stable vibration cavitation of the sonoactive microstructures moves the nucleic acid construct from an intravenous space into an interstitial space or into the cytoplasm.

In some embodiments, applying ultrasonic acoustic energy in (d) induces inertial cavitation of the sonoactive microstructures to disrupt the sonoactive microstructures. In some embodiments, applying ultrasonic acoustic energy in (d) induces inertial cavitation of the sonoactive microstructures to disrupt the sonoactive microstructures in a vasculature space and an extravascular space. In some embodiments, the extravascular spaces comprise an interstitial space, a subcutaneous space, intramuscular or a lymphatic space. In some embodiments, the extravascular spaces comprise an extravascular tissue. In some embodiments, the extravascular tissue comprises an interstitial space, a cytoplasmic space, a subcutaneous, a lymph tissues, muscular or combinations thereof.

In some embodiments, applying the ultrasonic acoustic energy of (d) induces formation of a pore in a membrane of the cell. In some embodiments, the formation of a pore in a membrane of the cell ranges from about 10 nm to about 10 um.

In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures.

In some embodiments, the nucleic acid construct comprises a miniplasmid backbone. As used herein, the term “miniplasmid (mpDNA)” refers to nucleic acid constructs that are smaller in size (i.e., contain fewer base pairs (bp)) than conventional plasmids or pDNA. In some embodiments, mpDNA constructs comprise a backbone smaller than 1 kb. In some embodiments, mpDNA constructs are smaller than 1000 bp excluding an expression cassette. In some embodiments, mpDNA constructs comprise a backbone smaller than 0.5 kb. In some embodiments, mpDNA constructs are smaller than 500 bp excluding an expression cassette. In some embodiments, the miniplasmid does not comprise a bacterial origin of replication. As used herein, the term “Nanoplasmid™” (e.g., Nanoplasmid sourced from Aldevron, Fargo, South Dakota.) refers to a small mpDNA construct that has a plasmid backbone that is less than 500 bp and does not contain an antibiotic resistance gene.

Miniplasmid DNA nucleic acid constructs can be utilized to deliver an expression cassette, a transgene, or a nonendogenous gene to cells in target cell-types, tissues or organs. In some embodiments, the miniplasmid comprises less than 1000 base pairs excluding an expression cassette. In some embodiments, the miniplasmid comprises less than 500 base pairs excluding an expression cassette. In some embodiments, the miniplasmid does not comprise antibiotic resistant genes. In some embodiments, the miniplasmid does not comprise a bacterial genome. In some embodiments, the miniplasmid comprises a therapeutic transgene and/or a regulatory element. In some embodiments, the miniplasmid is a nanoplasmid. In some embodiments, the miniplasmid construct enhances the expression of a nonendogenous gene or a therapeutic transgene when used in conjunction with the claimed methods and ultrasound acoustic profiles. In some embodiments, the nanoplasmid construct enhances the expression of a nonendogenous gene or a therapeutic transgene. In some embodiments, durability of expression of a protein encoded by the nucleic acid payload may be increased relative to expression of the same protein in a larger plasmid (e.g., a plasmid of greater than 2 kb in length, excluding the transgene). In some embodiments, durability of expression of a protein encoded by the nucleic acid payload may be increased relative to expression of the same protein in another nucleic acid construct.

In some embodiments, the nucleic acid construct is a miniplasmid (e.g., a construct comprising a backbone of less than 1000 bp or less than 500 bp) coupled to a nucleic acid payload.

In some embodiments, the nucleic acid payload comprises an expression cassette. In some embodiments, the expression cassette comprises a transgene. In some embodiments, the nucleic acid payload comprises a transgene (endogenous or non-endogenous). In some embodiments, the transgene comprises a therapeutic transgene. In some embodiments, inducing expression of the nucleic acid payload comprises inducing expression of the therapeutic transgene. In some embodiments, the transgene comprises a detectible marker. In some embodiments, the transgene comprises luciferase. In some embodiments, inducing expression of the nucleic acid payload comprises inducing expression of luciferase.

In some embodiments, a nucleic acid payload comprises a regulatory element such as a promoter, (e.g., APOE-ATT). In some embodiments, a total amount (e.g., dose) of DNA administered to a subject for purposes of sonoporation can range from 100 microgram to 200 mg.

In some embodiments, the therapeutic payload is a nonendogenous gene. In some embodiments, the nucleic acid payload is configured to perform gene augmentation, gene replacement, gene editing, gene knockdown, or gene knockout.

In some embodiments, the nucleic acid construct comprises one or more regulatory elements, such as a promoter, enhancer, ribosome binding site, or transcription termination signal. Examples of promoters contemplated herein include, but are not limited to, e.g., CMV promoter, UbC promoter, CAG promoter, EF-1a promoter, ApoE promoter, ApoE-AAT1 promoter, 3×SERP promoter, or P3-hybrid promoter. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising SERP. In some embodiments, the nucleic acid construct comprises a promoter sequence comprising P3.

In some embodiments, inducing expression of the nucleic acid payload comprises inducing production of RNA encoded by the payload. In some embodiments, inducing expression of the nucleic acid payload comprises inducing production of protein encoded by the payload.

In some embodiments, the payload comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides). In some embodiments, the nucleic acid construct is configured to perform gene augmentation, gene replacement, base editing, base knockdown, gene editing gene knockdown, or gene knockout. In some embodiments, delivering the nucleic acid payload to the target cell of the subject increases or decreases expression of a gene in the target cell.

In some embodiments, the payload comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease a regulatory protein or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR).

Sonoactive microstructures (also referred to as acoustic microspheres or “microbubbles”) contemplated herein include, but are not limited to, those used as ultrasonic imaging contrast agents. In some embodiments, the sonoactive microstructures comprise a phospholipid stabilized microstructure. In some embodiments, the phospholipid stabilized microstructure comprises a high molecular wight gas core, or a perflutran core. Examples of sonoactive microstructures include, but are not limited to, OPTISON (GE Healthcare), Sonazoid (GE Healthcare), or DEFINITY and Definity RT (Lantheus Medical Imaging, Inc). In some embodiments, the sonoactive microstructures are LUMASON (Bracco) (sulfur hexafluoride lipid-type A microspheres). In some embodiments, the sonoactive microstructures are SonoVue (sulfur hexafluoride microbubbles). In some embodiments, the sonoactive microstructures comprise a protein stabilized microstructure. In some embodiments, the sonoactive microstructures are Optison microbubbles.

The sonoactive microstructures can be administered prior to, after, or simultaneous (e.g., co-administered) with the administration of the nucleic acid construct (or nucleic acid payload). In some embodiments, the nucleic acid construct and the sonoactive microstructures are coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occurs sequentially. In some embodiments, the administering of the nucleic acid construct and the sonoactive microstructures occurs continuously.

In some embodiments, the nucleic acid construct is administered at a dosage of about 0.5 mg/kg to about 500 mg/kg. In some embodiments, about 2×10\hat{ }13 to about 3×10\hat{ }13 copies of the nucleic acid construct are administered to the subject. In some embodiments, each nucleic acid construct comprises a copy of of a transgene.

As used herein, concentrations of microstructures/mL refers to the concentration of the sonoactive microstructures in a pharmaceutical composition immediately prior to administration to the subject. In some embodiments, the sonoactive microstructures are administered at a concentration of about 5×10\hat{ }8 to about 1.2×10\hat{ }10 microstructures/mL. In some embodiments, the sonoactive microstructures are administered at a dosage of about 1-50 mL, for example 1 mL of a protein stabilized sonoactive microstructure (e.g., Optison). In some embodiments, the protein stabilized sonoactive microstructure (e.g., Optison) comprise a diameter of 3-4.5 micrometers. The sonoactive microstructures may be administered at a concentration of about 5M (million) to about 8M microstructures per mL. In some embodiments, the 1×10\hat{ }9 of phospholipid stabilized sonoactive microstructures (e.g., Sonazoid) are administered. In some embodiments, the phospholipid stabilized sonoactive microstructures (e.g., Sonazoid) comprise a diameter of 1-5 micrometers. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of at least 5×10\hat{ }8 microstructures per mL. In some embodiments, the sonoactive microstructures are administered at a concentration of up to 1.2×10\hat{ }10 microstructures/mL. In some embodiments, the sonoactive microstructures are administered at a concentration of 5×10\hat{ }8 to 8×10\hat{ }8 microstructures/mL.

In some embodiments, the nucleic acid construct and the sonoactive microstructures are mixed prior to being coadministered. In some instances, the sonoactive microstructures are mixed with the nucleic acid constructs before administering to the subject. In some instances, the sonoactive microstructures are mixed with the nucleic acid constructs along with additional buffers or agents such as saline or other biocompatible solutions with varying electrostatic charges and surface chemistries and ligands before administering to the subject. For example, Optison sonoactive microstructures can be mixed with a Nanoplasmid comprising APOE-Fluc and saline and administered together.

In some embodiments, the administering of the nucleic acid construct and the sonoactive microstructures is by intravenous administration or subcutaneous or intramuscular or intra-arterial or inter-osseus or direct organ puncture.

In some embodiments, after administering of the nucleic acid construct and sonoactive microstructures, the ultrasound acoustic energy is applied at the target cell, tissue, or organ.

Once the nucleic acid constructs are inside the target cell, expression of the nucleic acid payload is induced. In some embodiments, the nucleic acid payload comprises luciferase. In some embodiments, inducing expression of the nucleic acid payload using the miniplasmid construct comprises inducing expression inducing an average radiance of at least 2×10\hat{ }4 p/sec/cm2/sr. In some embodiments, inducing expression of the nucleic acid payload comprises inducing an average radiance of from about 2×10\hat{ }4 p/sec/cm2/sr to about 5×10\hat{ }5 p/sec/cm2/sr.

In some embodiments, inducing expression of the nucleic acid payload comprises inducing a flux of at least 10\hat{ }6 p/s. In some embodiments, inducing expression of the nucleic acid payload comprises inducing a flux of about 10\hat{ }6 p/s to about 10\hat{ }9 p/s.

In some embodiments, inducing expression of the nucleic acid payload comprises inducing a flux which is 2, 3, 4, or 5× greater than expression induced without repeating applying the ultrasonic acoustic energy at the first MI, and the applying the ultrasonic acoustic energy at the second MI.

In some embodiments, inducing expression of the nucleic acid payload comprises inducing expressing within about 3 to about 12 hours of administering the pay load. In some embodiments, inducing expression of the nucleic acid payload comprises inducing expressing within about 3 hours of administration. In some embodiments, inducing expression of the nucleic acid payload comprises inducing expressing within about 6 hours of administration. In some embodiments, inducing expression of the nucleic acid payload comprises inducing expressing within about 12 hours of administration.

Undesirable effects on living cells or tissues can occur due to ultrasound applications. In some embodiments, the present disclosure provides methods for improvement of gene transfection and not result in substantial DNA or cell damage in the target cells, tissues, or organs, using sonoporation by alternating ultrasonic acoustic energy between the first MI and the second MI. In some embodiments, the method does not result in substantial cellular damage to the target cell. In some embodiments, the method results in less than 1%, 5%, or 10% of target cells undergoing apoptosis.

Cellular damage can be detected using apoptotic biomarkers. For examples, in liver, detection of released hepatocellular transaminases, e.g., serum alanine aminotransferase (ALT) or aspartate aminotransferase (AST), can be an indicator of apoptotic hepatocytes. Additional apoptotic biomarkers comprise interleukin 6 (IL6) or B-cell lymphoma 2 (BCL2 or BCL2 apoptosis regulator). In some embodiments, the following biomarkers for cellular damage are not detected at apoptotic levels following delivering the nucleic acid payload to the target cell of the subject: ALT, AST, IL6, BCL2, or combinations thereof. In some embodiments, the following biomarkers for cellular damage are not clinically elevated following delivering the nucleic acid payload to the target cell of the subject: ALT, AST, IL6, BCL2, or combinations thereof. In some embodiments, the following biomarkers for cellular damage are not detected at apoptotic levels following delivering the nucleic acid payload to the target cell of the subject: ALT, AST, IL6, BCL2, or combinations thereof, and, optionally wherein the target cell is in a liver. In some embodiments, the following biomarkers for cellular damage are not clinically elevated following delivering the nucleic acid payload to the target cell of the subject: ALT, AST, IL6, BCL2, or combinations thereof, and, optionally wherein the target cell is in a liver. In some embodiments, the following biomarkers for cellular damage are not clinically elevated following delivering the nucleic acid payload to the target cell of the subject: creatinine levels in urine, albumin to creatine ratio in urine, creatinine levels in blood, a glomerular filtration rate, blood in urine, protein levels in urine, or an osmolality of urine, and, optionally wherein the target cell is in a kidney. In some embodiments, the following biomarkers for cellular damage are not clinically elevated following delivering the nucleic acid payload to the target cell of the subject: troponin levels in blood, or creatinine phospho kinase, and, optionally wherein the target cell is in a heart or skeletal muscle.

A sonoporation treatment using the methods described herein can be used to induce expression of a nucleic acid payload in a cell in a liver or a cell in a kidney.

A sonoporation treatment using the methods described herein can be used to treat a subject in need for gene therapy or enzyme replacement treatment. In another aspect, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher's (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

In some embodiments, the present disclosure provides methods of treating a subject having a liver condition with a therapeutic transgene. In some embodiments, the therapeutic transgene encodes one or more of: ATP7B; ABCB11; ABCB4; ATP8B1; TJP2; VWF; FVIII; FIX; F5; MAN2B1; GBA; SMPD1; CPS1; GDE/AGL; CTNS; SERPINA1; ASS1, and/or SLC25A13.

In some embodiments, the present disclosure provides methods of treating a subject having a liver condition with a therapeutic transgene. In some embodiments, the liver condition is Wilson's Disease, and the therapeutic transgene encodes ATP7B. In some embodiments, the liver condition is Cholestasis, progressive familial intrahepatic (PFIC1-4) and the therapeutic transgene encodes one or more of ABCB11, ABCB4, ATP8B1 and/or TJP2. In some embodiments, the liver condition is Von Willebrand Disease and the therapeutic transgene encodes VWF. In some embodiments, the liver condition is Hemophilia A, and the therapeutic transgene encodes FVIII. In some embodiments, the liver condition is Hemophilia B, and the therapeutic transgene encodes FIX. In some embodiments, the liver condition is Factor V Deficiency, and the therapeutic transgene encodes F5. In some embodiments, the liver condition is Alpha-Mannosidosis, and the therapeutic transgene encodes MAN2B1. In some embodiments, the liver condition is Gaucher's (glucocerebrosidase deficiency, glucocerebrosidosis), and the therapeutic transgene encodes GBA. In some embodiments, the liver condition is Niemann Pick Disease A/B, and the therapeutic transgene encodes SMPD1. In some embodiments, the liver condition is Carbamoylphosphate Synthetase I Deficiency, and the therapeutic transgene encodes CPS1. In some embodiments, the liver condition is Glycogen Storage Disease Type III, and the therapeutic transgene encodes GDE/AGL. In some embodiments, the liver condition is Cystinosis, and the therapeutic transgene encodes CTNS. In some embodiments, the liver condition is A1AT deficiency, and the therapeutic transgene encodes SERPINA1. In some embodiments, the liver condition is Citrullinemia Type I & II, and the therapeutic transgene encodes one or more of ASS1 and/or SLC25A13. In some embodiments, the methods comprise (a) administering to the subject a nucleic acid construct comprising the nucleic acid payload (e.g., a therapeutic transgene); (b) administering to the subject a plurality of sonoactive microstructures; and (c) administering a sonoporation treatment. In some embodiments, the sonoporation treatment comprises applying an ultrasonic acoustic energy to a liver at a first mechanical index (MI) that is less than 0.4; (d) applying an ultrasonic acoustic energy to the liver at a second MI that is greater than 0.4 and less than 2.0; In some embodiments, the method comprises repeating applying the ultrasonic acoustic energy at the first MI, and the applying the ultrasonic acoustic energy at the second MI a number of times. In some embodiments, the method comprises delivering the nucleic acid payload and the plurality of sonoactive microstructures systemically (e.g., by intravenous administration).

In some embodiments, provided herein is a method of treating a subject having Hemophilia A comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0).

In some embodiments, provided herein is a method of treating a subject having Wilson's Disease comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding ATP7B. In some embodiments, the nucleic acid construct and the plurality of sonoactive microstructures are administered systemically (e.g., by intravenous administration).

In one aspect, using the methods described herein, the present disclosure provides methods of treating a subject having a kidney condition. In some embodiments, the kidney condition treated is: Alport Syndrome, or Autosomal Dominant Polycystic Kidney Disease.

In some embodiments, the present disclosure provides methods of treating a subject having a kidney condition with a therapeutic transgene. In some embodiments, the therapeutic transgene encodes one or more of COL4A3, COL4A4, COL4A5, PKD1 and/or PKD2.

In some embodiments, the present disclosure provides methods of treating a subject having a kidney condition with a therapeutic transgene. In some embodiments, the kidney condition is Alport Syndrome, and the therapeutic transgene encodes one or more of COL4A3, COL4A4, and/or COL4A5. In some embodiments, the kidney condition is Autosomal Dominant Polycystic Kidney Disease, and the therapeutic transgene encodes one or more of PKD1 and/or PKD2. In some embodiments, the methods comprise (a) administering to the subject a nucleic acid construct comprising the nucleic acid payload; (b) administering to the subject a plurality of sonoactive microstructures; and (c) administering a sonoporation treatment. In some embodiments, the sonoporation treatment comprises applying an ultrasonic acoustic energy to a kidney at a first mechanical index (MI) that is less than 0.4; (d) applying an ultrasonic acoustic energy to the kidney at a second MI that is greater than 0.4 and less than 2.0.

In some embodiments, provided herein is a method of treating a subject having Alport Syndrome comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding COL4A3. In some embodiments, the therapeutic transgene comprises a nucleic acid sequence encoding COL4A4. In some embodiments, the therapeutic transgene comprises a nucleic acid sequence encoding COL4A5. In some embodiments, the nucleic acid construct and the plurality of sonoactive microstructures are administered systemically (e.g., by intravenous administration).

In some embodiments, provided herein is a method of treating a subject having Autosomal Polycystic Kidney Disease comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding PKD1. In some embodiments, the therapeutic transgene comprises a nucleic acid sequence encoding PKD2. In some embodiments, the nucleic acid construct and the plurality of sonoactive microstructures are administered systemically (e.g., by intravenous administration).

In another aspect, the present disclosure provides a kit to perform the methods described herein. In some embodiments, the kit comprises: (a) a first container comprising microbubbles for sonoporation; and (b) a second container comprising miniplasmids comprising a transgene and a mixture chamber (reservoir, syringe, Y-port, etc.).

In some embodiments, the miniplasmid further comprises an expression cassette. As used herein, an expression cassette comprises nucleic acid sequences encoding nucleic acid payload, e.g., an expression cassette comprising a transgene. The expression cassette further comprises a regulatory element such as a promoter, enhancer, ribosome binding site, or transcription termination signal.

In some embodiments, the first container and second container are configured to induce the expression of the transgene in the target cell of the subject within 20 hours after the transfection.

In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell at the low MI and the high MI. In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell at the low MI and the high MI.

In some embodiments, the method further includes increasing expression of the nucleic acid payload by increasing the dosage of the nucleic acid payload administered to the subject. In some embodiments, the method further includes increasing expression of the nucleic acid payload by increasing the dosage of the nucleic acid payload administered to the subject in a linear manner. In some embodiments, the method further includes increasing expression of the nucleic acid payload by administering at least 5, 50, 250, or 500 μg of the nucleic acid payload to the subject.

In some embodiments, ALT is not detected at levels exceeding 60, 65, 70, 75, 80, 85, 90, 95, 100, 125, 150, 175, or 200 U/L following delivering the nucleic acid payload to the target cell of the subject. In some embodiments, AST is not detected at levels exceeding 225, 250, 275, or 300 U/L following delivering the nucleic acid payload to the target cell of the subject. In some embodiments, IL6 is not detected at levels exceeding 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, or 6 μg/mL following delivering the nucleic acid payload to the target cell of the subject.

In some embodiments, the kit further comprises instructions for software and hardware directions for the safe and effective operation of an ultrasound machine sufficient to disrupt the sonoactive microstructures to generate the sonoporation processes which include but are not limited to the following: disrupting the microstructures, inducing inertial and stable cavitation, promoting endocytosis and inter-endothelial gap formation, microstreaming at cell surfaces, thereby increasing transfection of a nucleic acid payload to a cell. In some embodiments, the instructions described methods for improvement of gene transfection using sonoporation by applying alternating ultrasonic acoustic energy between a first MI then a second MI. In some embodiments, the kit further comprises instructions for administration of the first container and the second container.

The present disclosure provides ultrasound systems comprising computer systems that are programmed to implement methods of the disclosure. The ultrasound systems 200 may be operably connected to one or more ultrasound transducers 211 controlled by a computer system 201 one or more computer processers 204 which may comprise one or more computer readable medium/media 205 which comprise instructions configured to cause the ultrasound systems to perform the methods of the present disclosure. The ultrasound systems 200 and/or the computer processers 204 may be in communication with the cloud 207 or other remote server which enable the remote operation and control of the ultrasound systems 200 and performance of the methods disclosed herein. The computer system 201 can be an electronic device of a user or a computer system that is remotely located with respect to the electronic device. The electronic device can be a mobile electronic device. The computer system includes a central processing unit (CPU, also “processor” and “computer processor” herein), which can be a single core or multi core processor, or a plurality of processors for parallel processing. The computer system also includes memory or memory location 206 (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

Aspects disclosed herein provide a system (e.g., ultrasound systems) comprising: an ultrasound transducer configured to apply ultrasound acoustic energy to a subject at a plurality of mechanical indexes; a computer system comprising a computer processor and a computer-readable medium, wherein the computer system is configured to implement a method of applying ultrasonic acoustic energy to a target cell of the subject, the method comprising: applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0), wherein the subject has been administered a nucleic acid construct comprising the nucleic acid payload, wherein the nucleic acid construct is a miniplasmid, and a plurality of sonoactive microstructures. In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the nucleic acid construct is a plasmid that is less than or equal to 500 base pairs in length excluding an expression cassette, or wherein the wherein the nucleic acid construct is a miniplasmid. In some embodiments, applying the ultrasonic acoustic energy of at the first MI and applying the ultrasonic acoustic energy of at the second MI are repeated at least twice. In some embodiments, applying the ultrasonic acoustic energy of at the first MI and applying the ultrasonic acoustic energy at the second MI are repeated from 4 to 18 times. In some embodiments, applying the ultrasonic acoustic energy of at the first MI and applying the ultrasonic acoustic energy at the second MI are repeated from 6 to 12 times. In some embodiments, applying the ultrasonic acoustic energy of at the first MI and applying the ultrasonic acoustic energy at the second MI are repeated from 8 to 10 times. In some embodiments, the second MI ranges from about 1.4 to about 2.0. In some embodiments, applying the ultrasound acoustic energy of at the second mechanical index induces formation of a pore in a membrane of the cell. In some embodiments, applying the ultrasound acoustic energy of at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy of d. comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of about 2.3 μs. In some embodiments, the method includes repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI.

The systems (e.g., ultrasound systems) disclosed herein may be controlled or operated by a computer comprising a computer-readable medium configured to implement a method of applying ultrasonic acoustic energy to a target cell of the subject, the method comprising: applying an ultrasonic acoustic energy to the target cell at a first mechanical index (MI) that is up to 0.4; and applying an ultrasonic acoustic energy to the target cell at a second MI that is greater than 0.4 and up to 2.0, wherein the subject has been administered (1) a nucleic acid construct comprising a nucleic acid payload, wherein the nucleic acid construct is a miniplasmid, and (2) a plurality of sonoactive microstructures. The computer readable medium of claim 153, wherein an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the miniplasmid is less than or equal to 500 base pairs in length excluding an expression cassette. In some embodiments, applying the ultrasonic acoustic energy at the first MI and applying the ultrasonic acoustic energy at the second MI are repeated at least twice. In some embodiments, applying the ultrasonic acoustic energy at the first MI and applying the ultrasonic acoustic energy at the second MI are repeated from 4 to 18 times. In some embodiments, applying the ultrasonic acoustic energy at the first MI and applying the ultrasonic acoustic energy at the second MI are repeated from 6 to 12 times. In some embodiments, applying the ultrasonic acoustic energy at the first MI and applying the ultrasonic acoustic energy at the second MI are repeated from 8 to 10 times. In some embodiments, the second MI ranges from about 1.4 to about 2.0. In some embodiments, applying the ultrasound acoustic energy at the second mechanical index induces formation of a pore in a membrane of the cell. In some embodiments, applying the ultrasound acoustic energy at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy at the second MI comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of about 2.3 μs. In some embodiments, the instructions comprise repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, wherein the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., hand-held device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, concentration of the analyte of interest. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit.

In some aspects, the disclosed provides quality control methods or methods to assess a risk associated with a food, with a hospital, with a clinic, or any other location where the presence of a bacterium poses a certain risk to one or more subjects. In many instances, systems, platforms, software, networks, and methods described herein include a digital processing device, or use of the same. In further embodiments, the digital processing device includes one or more hardware central processing units (CPUs), i.e., processors that carry out the device's functions, such as the automated sequencing apparatus disclosed herein or a computer system used in the analyses of a plurality of nucleic acid sequencing reads from samples derived from a food processing facility or from any other facility, such as a hospital a clinical or another. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable digital processing device that can either be deployed to a food processing facility, or is used within said food processing facility to process and analyze a variety of nucleic acids from a variety of samples.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280× about 720 pixels or at least about 1920× about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

In many aspects, the systems, platforms, software, networks, and methods disclosed herein include one or more non-transitory computer readable storage media encoded with a program including instructions executable by the operating system of an optionally networked digital processing device. For instance, in some aspects, the methods comprise creating data files associated with a plurality of sequencing reads from a plurality of samples associated with a food processing facility. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

In some embodiments, after a first body of the 3D object is produced, the movable stage is removed from the actuator system. The 3D object may then continue to further processing steps, such as a perfusion sequence as described herein. The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Definitions

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

### EXAMPLES

The following examples are included for illustrative purposes only and are not intended to limit the scope of the invention.

### Example 1: Generation of Miniplasmid for Sonoporation

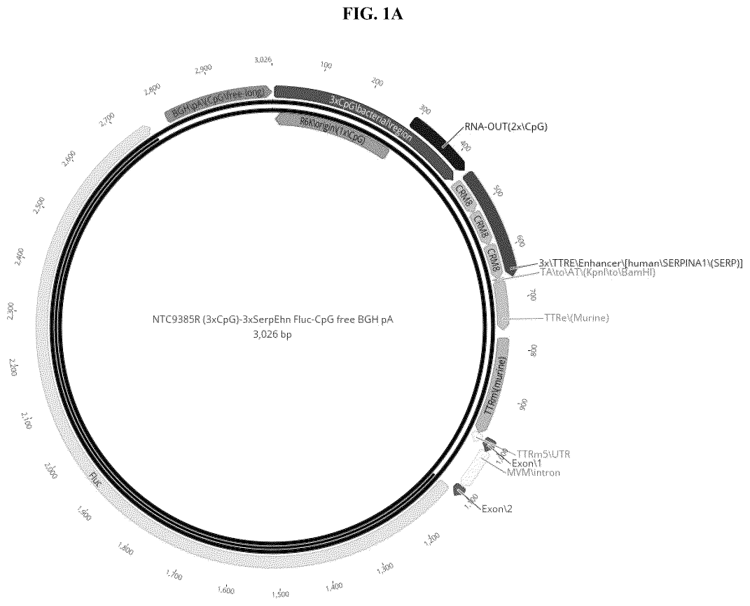

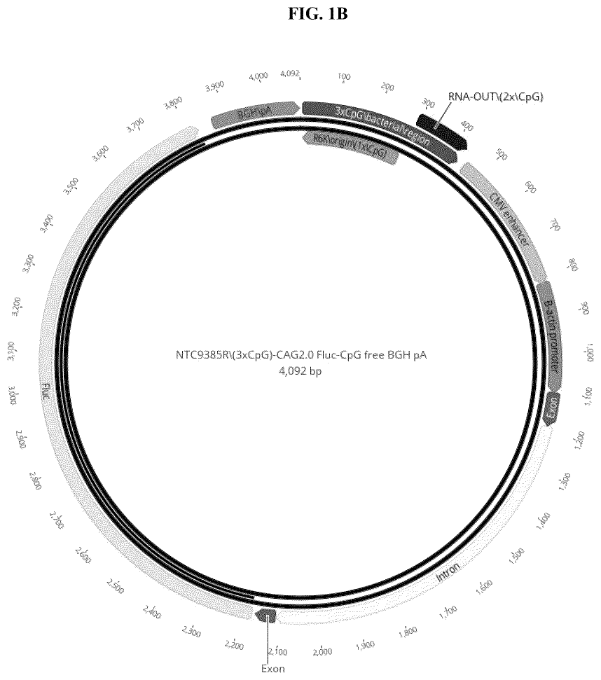

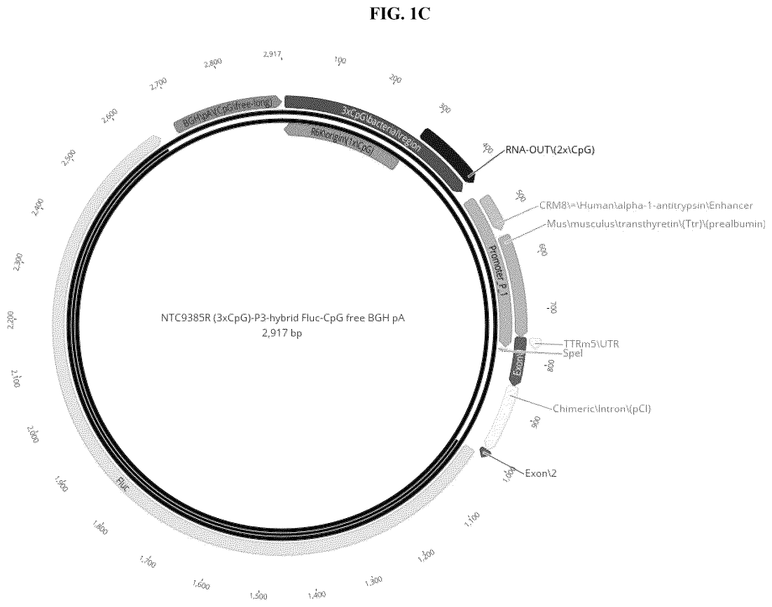

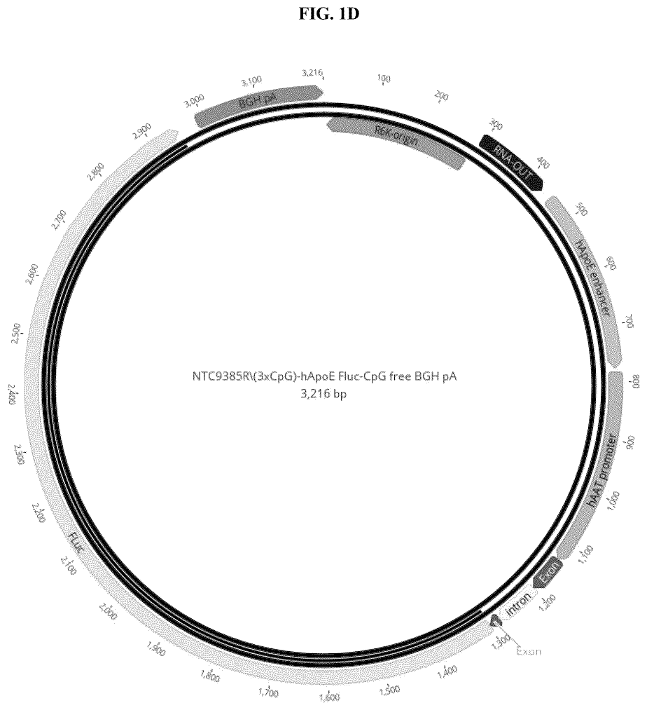

In this experiment, generation of miniplasmids for transfection was performed. Briefly, a miniplasmid vector backbone, e.g., a nanoplasmid, was used. Nanoplasmids were generated/purchased from Aldeveron (Fargo, SD). Wildtype firefly luciferase was used as a reporter gene in this experiment and was located under a promoter sequence. The nucleic acid constructs included: CAG-Fluc, ApoE-AAT-Fluc, 3×SERP-Enh-TTR-Fluc, and P3-hybrid-Fluc. Nanoplasmid vector maps are shown in FIG. 1.

### Example 2: Optimization of Expression and Durability of Gene Therapy in Rat Liver

This experiment evaluated the transfection and expression of the reporter gene luciferase in a rat liver.

### Experimental Conditions and Protocols:

Twenty Sprague Dawley rats were studied. All animals were anesthetized with 2% Isoflurane and the abdomen was shaved, and a depilatory agent was applied. The injectate comprised of 1 mL Optison and 250 μL of a nucleic acid payload comprising DNA (1.125 mg) of one of the following nanoplasmids: a nanoplasmid comprising a promoter sequence ApoE-AAT, CAG, 3×SERP, or P3; and each nanoplasmid comprising luciferase (e.g., nanoplasmids generated in Example 1). The nucleic acids payloads were diluted with 750 μL PBS with an estimated dead space of about 75 μL. The solution was intravenously infused via a tail vein over 70 seconds. An acoustic contact agent (Aqua gel) was directly applied to the abdominal surface and the was applied to the upper abdominal skin surface of the rat. The acoustic parameters included the following:
The Low MI operated at an MI or 0.09.
The High MI mode operated at an MI of 1.4.

Ultrasound was delivered at a frequency of 9.3 MHz.

The therapeutic procedure was administered as follows:
Simultaneous with the tail vein infusion, the ultrasound transducer was placed on the abdomen and Low MI ultrasound imaging (0.09) of the liver was initiated for 20 seconds.
After 21 seconds, a pulse of a High MI of 1.4 was applied for a pulse duration of 0.98 μs.
After the High MI pulse, a Low MI imaging (0.09) was continued, and the High MI pulse was implemented every 10 seconds, 8 times (total of 90 seconds).

In vivo bioluminescence imaging (IVIS) was performed at 24, 48, 72 and 144 hours. The bioluminescence values are reported as Average Radiance (photons/sec/cm²/steradians).

### Results:

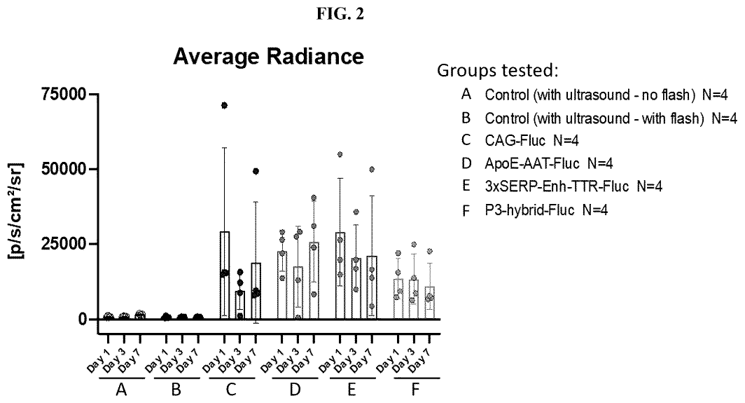

As shown in FIG. 2, Group A and B were control groups. Group C, D, E, and F (N=4 each group) received the nucleic acid payload comprising the DNA in this order: CAG-Fluc, ApoE-AAT-Fluc, 3×SERP-Enh-TTR-Fluc, and P3-hybrid-Fluc. The average radiance was recorded within IVIS in (photons/sec/cm²/steradian). As noted, the control animals did not exhibit bioluminescence, and groups D and E revealed stable, average radiance at 144 hours with increased variability noted in groups C and F.

FIG. 2 depicts quantitative results of nucleic acid transfection and expression from In Vivo Imaging System (IVIS) using bioluminescence imaging (BLI) of rat liver using nucleic acid payloads comprising CAG-Fluc, ApoE-AAT-Fluc, 3×SERP-Enh-TTR-Fluc, and P3-hybrid-Fluc.

### Example 3: Optimization of Expression and Durability of Gene Therapy in Mouse Liver—Study I

In this experiment, the kinetics transfection and expression of the reporter gene luciferase were investigated in a mouse liver.

### Experimental Conditions and Protocols:

12 C57BL/6 mice were studied. All animals were anesthetized with 2% Isoflurane and the abdomen was shaved, and a depilatory agent was applied. The ApoE-ATT/luciferase nanoplasmid generated in the Example 1 was used. The injectate comprises a total injectate volume of 240 μL (157.5 μg of ApoE-AAT with luciferase nanoplasmids) in 35 μL with 95 μL of PBS and 120 μL of Optison, with estimated dead space of about 50 μL. The total volume was intravenously infused via a tail vein over 70 seconds and external ultrasound transducer. An acoustic contact agent (Aqua gel) was directly applied to the abdominal surface and the ultrasound acoustic energy was applied to the upper abdominal skin surface of the rat. The acoustic parameters included the following:
The Low MI operated at an MI or 0.09 or 0.3.
The High MI mode operated at a MI of 1.5.

Ultrasound was delivered at a frequency of 9.3 MHz.

The therapeutic procedure was administered as follows:
Simultaneous with the tail vein infusion, Low MI imaging (0.09) of the liver was initiated for the initial 20 seconds following the infusion.
At 21 seconds, a pulse of a High MI of 1.5 was applied for a pulse duration of 2.28 μsec.
After the High MI mode, a Low MI imaging (0.09) was continued, and the High MI was implemented every 10 seconds for 9 times (total of 100 seconds)

Additional studies included variations on the above acoustic parameters where the low MI was 0.09 and other mice received a Low MI of 0.3.

The liver-based, protein kinetics post sonoporation were characterized using IVIS with measurements initiated at 3 hours and sequentially recorded at 6, 12, 18, 24, 30, 48 and 72 hours.

### Results:

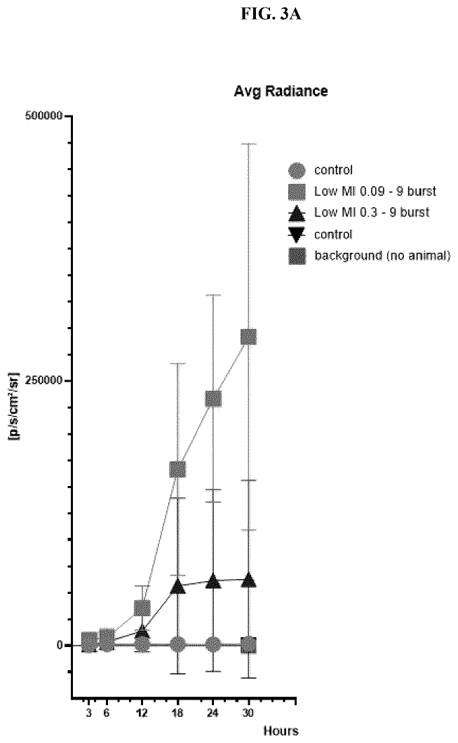

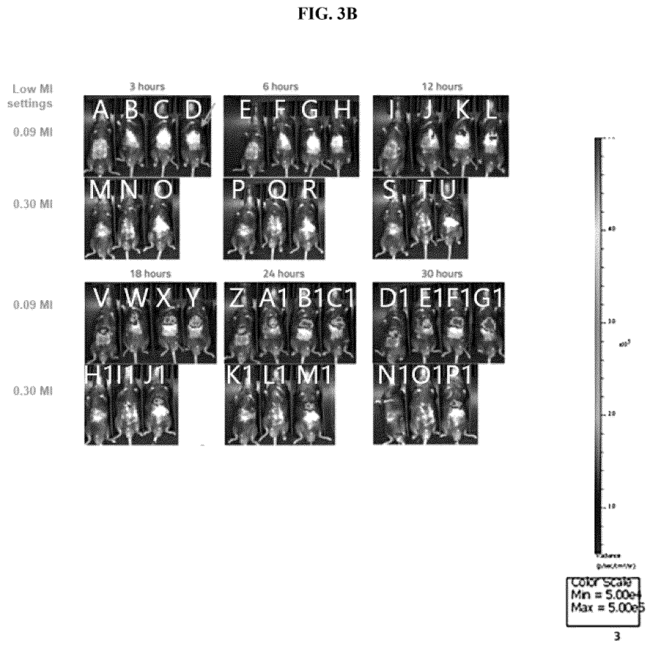

As shown in FIG. 3A, the background control groups did not reveal any recorded bioluminescence. Based on timed kinetic data, the initial IVIS scan performed at 3 hours revealed the presence of a luciferase signal. The bioluminescence signal levels increased at 6, 12, 18, 24 hours, with the peak signal noted at 30 hours in 2 animals that received Low MI (0.09) treatments (all other conditions were constant). The bioluminescence signal was substantially lower in the animals that received a reduced Pulse numbers (N=4 versus vs. N=9) or were treated with elevated Low MI at 0.3 or received inadequate tail vein injections. The bioluminescence imaging results are shown in FIG. 3B.

FIGS. 3A and 3B depicts quantitative results of nucleic acid transfection and expression (kinetic study) from IVIS using BLI of mouse liver from Study I with a nucleic acid payload comprising ApoE-AAT-Fluc. FIG. 3A shows a graph of average radiance measured as compared to a control. FIG. 3B shows the same In Vivo Imaging System (IVIS) using bioluminescence imaging (BLI). In FIG. 3B it observed that: there is no observable fluorescence in panels A-C; there is blue/green fluorescence is the mouse liver in panel D; there is no observable fluorescence in panels E-G; there is there is blue/green fluorescence is the mouse liver in panel H, with an increase in blue/green fluorescence in panel H relative to panel D; there is a small amount of blue fluorescence in panel I in the mouse liver; there is observable blue/green fluorescence is the mouse liver in panels J-L, with an increase in blue/green fluorescence in panel L relative to panel H; there is no observable fluorescence in panels M-T; there is blue/green fluorescence in the mouse liver in panel U; there is blue/green fluorescence in in the mouse liver in panels V-W; there is green/red fluorescence in in the mouse liver in panels X-Y; there is blue/green fluorescence in in the mouse liver in panel Z, with an increase in blue/green fluorescence in panel A relative to panel V; there is green/red fluorescence in in the mouse liver in panels A1-C1, with an increase in red/green fluorescence in panels A1-C1 relative to panels W-Y; there is blue/green fluorescence in the mouse liver in panel D1; there is green/red fluorescence in in the mouse liver in panels E1-G1; there is no observable fluorescence in panels H1, K1, or N1; there is blue/green fluorescence in the mouse liver in panels I1 and L1, with an increase in the blue/green fluorescence in panel L1 relative to panel I1; there is red/green fluorescence panels J1 and M1, with an fluorescence in panel M1 relative to panel J1; and there is red/green fluorescence panels P1 and M1, with an fluorescence in panel P1 relative to panel M1. Blue fluorescence is indicative of about 10*10≡p/sec/cm²/sr in fluorescence intensity; blue/green fluorescence is indicative of about 20*10\hat{ }6 p/sec/cm²/sr in fluorescence intensity; green fluorescence is indicative of about 30*_{10\hat{ }6} p/sec/cm²/sr in fluorescence intensity; yellow fluorescence is indicative of about 40*10\hat{ }6 p/sec/cm²/sr in fluorescence intensity; and red fluorescence is indicative of about 50*10\hat{ }6 p/sec/cm²/sr in fluorescence intensity. Increased fluorescence is indicative of a higher degree of luciferase expression

### Example 4: Optimization of Expression and Durability of Gene Therapy in Mouse Liver—Study II

In this experiment, the transfection and expression of the reporter gene luciferase were investigated in a mouse liver.

### Experimental Conditions and Protocols:

12 BalbC mice were studied. All animals were anesthetized with 2% Isoflurane and the abdomen was shaved, and a depilatory agent was applied. The nanoplasmids generated in the Example 1 was used. The injectate comprised of a total injectate volume of 300 μL (157.5 μg of ApoE-AAT with luciferase nanoplasmids in 35 μL with 115 μL of PBS and 150 μL of Optison, with estimated dead space of about 50 μL). The total volume was intravenously infused via a tail vein over 3 seconds and external ultrasound transducer. An acoustic contact agent (Aqua gel) was directly applied to the abdominal surface and the transducer was applied to the upper abdominal skin surface of the rat. The acoustic parameters included the following:
The low MI operated at an MI or 0.05-0.07.
The High MI mode operated at an MI of 0.8.

Ultrasound was delivered at a frequency of 9.3 MHz.

The therapeutic procedure was administered as follows:
Simultaneous with the tail vein infusion, a High MI Pulse was administered at 8 second intervals at a repetition rate of 4 or 9 or 18 sequences (32, 72 or 144 seconds, respectively), for a pulse duration of 2.28 μsec.
The Low MI imaging remained at (0.05-0.07) throughout the therapeutic session, and was administered between the high MI pulses.

IVIS was performed at 24, 48, 72 and hours.

### Result:

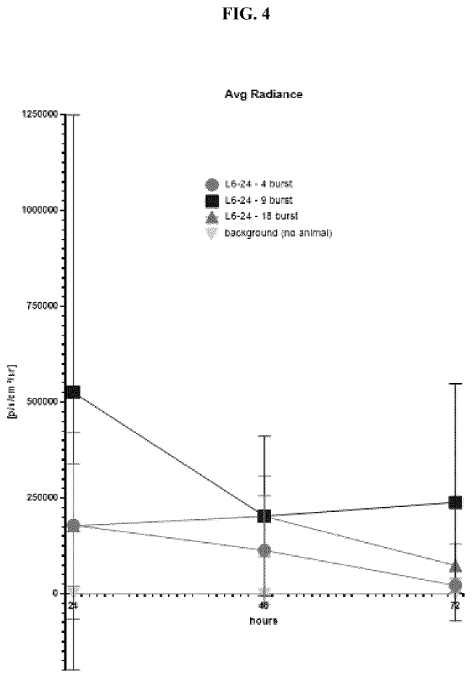

As shown in FIG. 4, the background did not reveal bioluminescence. The most stable bioluminescence result was recorded in the animals that received 9 pulses (as indicated in FIG. 4) at 8 second intervals; whereas the animals that received either 4 Pulse sequences or 18 Pulse sequences at 8 second intervals did not reveal a stable bioluminescence pattern at 72 hours, indicating significantly reduced expression of luciferase at 72 hours. The animals that received 9 pulses experienced a stable illuminance response, suggesting that the expression of luciferase was maintained.

### Example 5: Expression and Durability of Gene Therapy in Mouse Kidney

In this experiment, efficacy and durability of gene therapy post sonoporation were investigated in mouse kidney.

### Experimental Conditions and Protocols:

8 BalbC mice were studied. All animals were anesthetized with 2% Isoflurane and the abdomen and lower back area were shaved, and a depilatory agent was applied. The nanoplasmid CAG generated in the Example 1 was used. The injectate consisted of a total injectate volume of 300 μL (157.5 μg of CAG with luciferase nanoplasmids in 35 μL with 115 μL of PBS and 150 μL of Optison, with estimated dead space of about 50 μL). The total volume was intravenously infused via a tail vein over 3 seconds and external ultrasound transducer.

An acoustic contact agent, Aqua gel, was directly applied to the left lateral abdominal surface and the transducer was applied to the left upper abdominal skin surface with a focus on the left kidney area. Imaging permitted clear acoustic visualization of the left kidney. All treated animals received DNA (CAG). The acoustic parameters included the following:
The low MI operated at an MI or 0.05-0.07.
The High MI mode operated at 0.8 MI with a pulse duration of approximately 2 microseconds.

Ultrasound was delivered at a frequency of 9.3 MHz.

The therapeutic procedure was administered as follows:
Simultaneous with the tail vein infusions, High MI Pulse was initially administered every 3 seconds with a repetition rate of 10 Pulse sequences (total of 27 seconds). The Low MI imaging remained at (0.05-0.07) throughout the therapeutic session.

In vivo bioluminescence imaging (IVIS) was performed at 17 and 36 hours.

### Result:

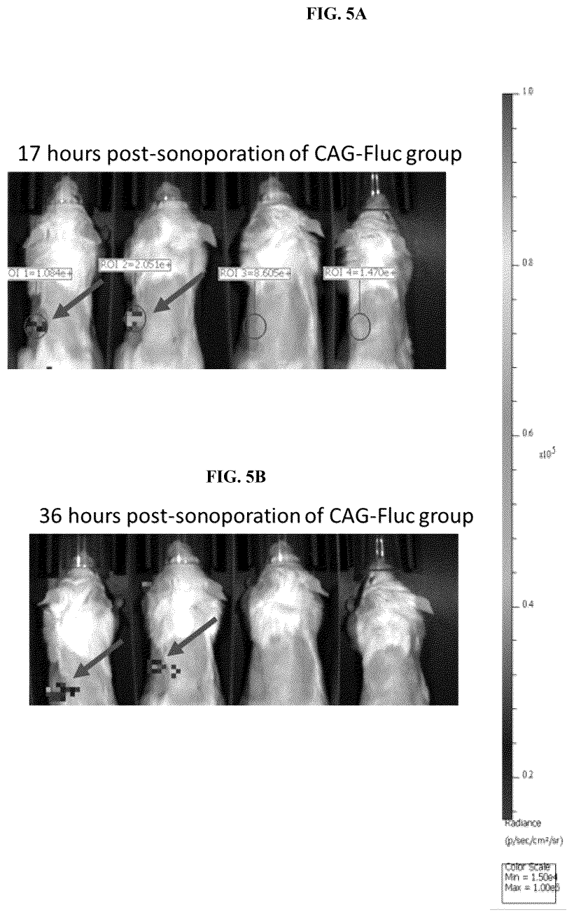

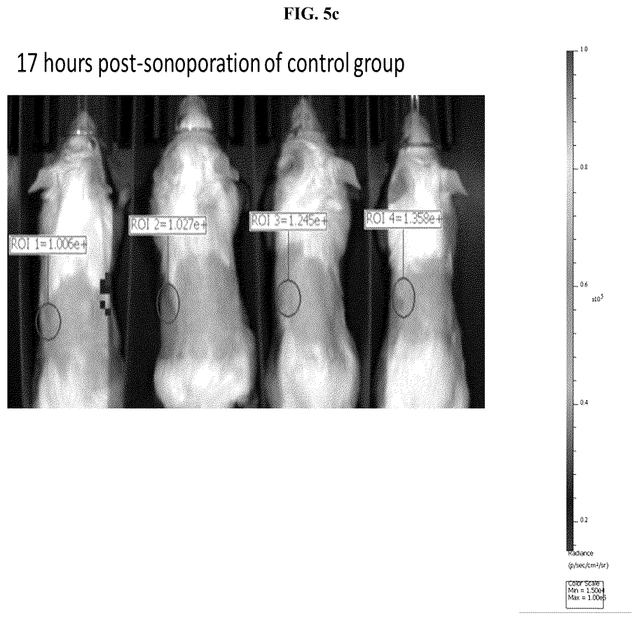

As shown in FIG. 5A, at 17 hours post sonoporation, bioluminescence was recorded in 2 of the 4 animals (left 2 mice) notably in the left lateral region. Both animals received sonoporation treatment directed to the left kidney and were imaged in a short axis plane; whereas the 2 mice on the right side of the image were imaged in the long-axis plane. FIG. 5B shows that, at 36 hours post-sonoporation, the expression of reporter gene was still observed. As shown in FIG. 5C, in the control animals, there was no bioluminescence noted in the left lateral region. Table 1 below shows quantitative result of this experiment.

FIGS. 5A-5C depict imaging results from IVIS of mouse kidney after receiving CAG-Fluc at 17 hours and 36 hours post-treatment, respectively. FIG. 5A shows blue colored fluorescence in the leftmost image of the kidney of the subject. FIG. 5A shows blue/green colored fluorescence in the second leftmost image of the kidney of the subject. FIG. 5A shows no fluorescence in the third leftmost image of the kidney of the subject. FIG. 5A shows no fluorescence in the third leftmost image of the kidney of the subject. FIG. 5B shows blue/green colored fluorescence in the rightmost image of the kidney of the subject. FIG. 5B shows blue/green colored fluorescence in the second leftmost image of the kidney of the subject. FIG. 5B shows no fluorescence in the third leftmost image of the kidney of the subject. FIG. 5B shows no fluorescence in the rightmost image of the kidney of the subject. Blue fluorescence is indicative of about 10*10\hat{ }5 p/sec/cm²/sr in fluorescence intensity; blue/green fluorescence is indicative of about 20*10\hat{ }6 p/sec/cm²/sr in fluorescence intensity; green fluorescence is indicative of about 30*10\hat{ }6 p/sec/cm²/sr in fluorescence intensity; yellow fluorescence is indicative of about 40*10\hat{ }6 p/sec/cm²/sr in fluorescence intensity; and red fluorescence is indicative of about 50*10\hat{ }6 p/sec/cm²/sr in fluorescence intensity. Increased fluorescence is indicative of a higher degree of luciferase expression. FIG. 5C depicts imaging results from IVIS of mouse kidney from the control group. FIG. 5C shows no fluorescence in the leftmost image of the kidney of the subject. FIG. 5C shows no fluorescence in the second leftmost image of the kidney of the subject. FIG. 5C shows no fluorescence in the third leftmost image of the kidney of the subject. FIG. 5C shows no fluorescence in the rightmost image of the kidney of the subject. Blue fluorescence is indicative of about 10*10\hat{ }5 p/sec/cm²/sr in fluorescence intensity; blue/green fluorescence is indicative of about 20*10\hat{ }6 p/sec/cm²/sr in fluorescence intensity; green fluorescence is indicative of about 30*10\hat{ }6 p/sec/cm²/sr in fluorescence intensity; yellow fluorescence is indicative of about 40*10\hat{ }6 p/sec/cm²/sr in fluorescence intensity; and red fluorescence is indicative of about 50*10\hat{ }6 p/sec/cm²/sr in fluorescence intensity. Increased fluorescence is indicative of a higher degree of luciferase expression.

TABLE 1
Quantification of IVIS readout.

| | | Total | Avg | |
|---|---|---|---|---|
| | Time and mouse | Flux | Radiance | Area |
| Delivery | number | [p/s] | [p/s/c [symbol] | [cm²] |
| SHORT_{—} | 17 hours - mouse 1 | 1.08E+05 | 1.53E+04 | 5.63E−01 |
| AXIS | 17 hours - mouse 2 | 2.05E+05 | 2.58E+04 | 6.33E−01 |
| DELIVERY | ave | 1.57E+05 | 2.06E+04 | 5.98E−01 |
| | st dev | 6.84E+04 | 7.40E+03 | 4.97E−02 |
| LONG_AXIS | 17 hours - mouse 3 | 8.61E+03 | 1.22E+03 | 5.63E−01 |
| DELIVERY | 17 hours - mouse 4 | 1.47E+04 | 2.08E+03 | 5.63E−01 |
| | ave | 1.17E+04 | 1.65E+03 | 5.63E−01 |
| | st dev | 4309.816 | 609.526 | 0 |
| SHORT_{—} | 36 hours - mouse 1 | 1.54E+05 | 2.05E+04 | 5.98E−01 |
| AXIS | 36 hours - mouse 2 | 1.24E+05 | 1.56E+04 | 6.33E−01 |
| DELIVERY | ave | 1.39E+05 | 1.80E+04 | 6.15E−01 |
| | st dev | 2.13E+04 | 3.49E+03 | 2.48E−02 |
| LONG_AXIS | 36 hours - mouse 3 | 1.67E+04 | 2.37E+03 | 5.63E−01 |
| DELIVERY | 36 hours - mouse 4 | 2.36E+04 | 3.15E+03 | 5.98E−01 |
| | ave | 2.02E+04 | 2.76E+03 | 5.80E−01 |
| | st dev | 4.87E+03 | 5.50E+02 | 2.49E−02 |
| 17 HOURS | control 1 | 1.01E+04 | 1.27E+03 | 6.33E−01 |
| | control 2 | 1.03E+04 | 1.37E+03 | 5.98E−01 |
| | control 3 | 1.25E+04 | 1.76E+03 | 5.63E−01 |
| | control 4 | 1.36E+04 | 1.92E+03 | 5.63E−01 |
| | ave | 1.16E+04 | 1.58E+03 | 5.89E−01 |
| | st dev | 1.71E+03 | 3.13E+02 | 3.37E−02 |

[symbol]

indicates data missing or illegible when filed

Overall, this data shows that the nucleic acid payload comprising the nanoplasmid construct comprising luciferase was delivered to kidney using alternating MI protocol.

Table 2 below provides summary of parameters used in Examples 2-5.

TABLE 2
Summary of parameters used in Examples 2-5.

| Figure and | Example 2 | | Example 3 | | Example 4 | | Example 5 | |
|---|---|---|---|---|---|---|---|---|
| description | Rat liver | | Mice Liver I | | Mice Liver II | | Mice Kidney I | |
| Species/#number | Rats (SD), | | Mice, | | Mice, | | Mice, | |
| Conditions | 20 rats | | C57BL/6 | | BalbC | | BalbC | |
| | All | | Total 12 | | Total 12 | | Total 8 | |
| | ultrasound | | mice: | | mice: | | mice: | |
| | parameters | | 4 mice at low | | 4 mice at 4 | | 4 treated. | |
| | were | | MI of 0.09 | | pulses | | Kidneys | |
| | constant | | 4 mice at | | 4 mice at 9 | | were | |
| | (condition | | Low MI of | | pulses | | imaged in | |
| | described | | 0.3 | | 4 mice at 18 | | the short | |
| | below) | | 4 mice at low | | pulses | | axis (N = 2) | |
| | | | MI of 0.03 | | | | and in the | |
| | | | (and 4 | | | | long axis | |
| | | | pulses) | | | | (N = 2) | |
| | | | | | | | N = 4 | |
| | | | | | | | control | |
| | | | | | | | animals | |
| Target organ | liver | | liver | | liver | | kidney | |
| Ultrasound | Optison | | Optison | | Optison | | Optison | |
| agent | 50% v/v PBS | | 50% v/v PBS | | 50% v/v PBS | | 50% v/v PBS | |
| DNA 4.5 mg/mL | ApoE-ATT, | | ApoE-AAT | | ApoE-AAT | | CAG | |
| (concentration) | CAG, | | Total | | Total | | Total | |
| APOE | 3xSERP and | | delivered | | delivered | | delivered | |
| CAG | P3 used. | | about | | about | | about | |
| SERP | Total | | 157.5 μg | | 157.5 μg | | 157.5 μg | |
| P3 | delivered | | DNA | | DNA | | DNA | |
| | about | | | | | | | |
| | 1.125 mg | | | | | | | |
| | DNA | | | | | | | |
| Injectate volume | Total of | | Total 240 | | Total 300 | | Total 300 | |
| and components | 2 mL; | | μL; | | μL | | μL; | |
| | deadspace | | deadspace | | deadspace | | deadspace | |
| | about 75 μL; | | about 50 μL; | | about 50 μL; | | about 50 μL; | |
| | 1 mL Optison | | 120 μL | | 150 μL | | 150 μL | |
| | and 250 μL | | Optison and | | Optison and | | Optison and | |
| | of DNA with | | 35 μL DNA | | 35 μL DNA | | 35 μL DNA | |
| | 750 μL PBS | | and 95 μL | | and 115 μL | | and 115 μL | |
| | | | of PBS | | of PBS | | of PBS | |
| Tail vein volume | 2 | ml | 240 | | 300 | | 300 | |
| | | | microliters | | microliters | | microliters | |
| Infusion | 70 sec | | 70 sec | | 3 sec bolus | | 3 sec bolus | |
| duration | infusion | | infusion | | | | | |
| Duration of | 90 | sec | 90 | sec | 4 pulses @ | | 10 pulses at | |
| treatment | | | | | 8 sec total | | 3 seconds = | |
| | | | | | time 32 sec | | 30 sec | |
| | | | | | 9 pulses @ | | | |
| | | | | | 8 sec total | | | |
| | | | | | time 72 sec | | | |
| | | | | | 18 pulses | | | |
| | | | | | @8 sec total | | | |
| | | | | | time 144 sec | | | |
| Low MI | 0.09 | | 0.09 | | 0.05-0.07 | | 0.05-0.07 | |
| HI MI | 1.4 | | 1.5 | | 0.8 | | 0.8 | |
| Frequency | 9.3 | MHz | 9.3 | MHz | 9.3 | MHz | 9.3 | MHz |
| Pulses (#) total | 8 | | 4 and 9 | | 4, 9 and 18 | | 10 | pulses |
| Time of 1^{st} Pulse | 20 | seconds | 20 | seconds | 8 | seconds | Immediate | |
| after infusion | | | | | | | (no delay) | |
| High MI Pulse | 10 | seconds | 10 | seconds | 8 | seconds | 3 | seconds |
| timed sequences | | | | | | | | |

### Example 6: Dose-Responsive Delivery in Mouse Liver

In this experiment, the effect of transgene dose on expression in the mouse liver was investigated. The injectate included 5 μg, 50 μg, 250 μg, or 500 μg of a luciferase nanoplasmid. Sonoporation was performed according to the methods described herein. Gene expression was analyzed by IVIS.

The same experimental procedure is performed as described in the previous example for the mouse liver. Ultrasound was applied at 9.3 MHz, a low MI of 0.1-0.4, and a high MI of 1.4.

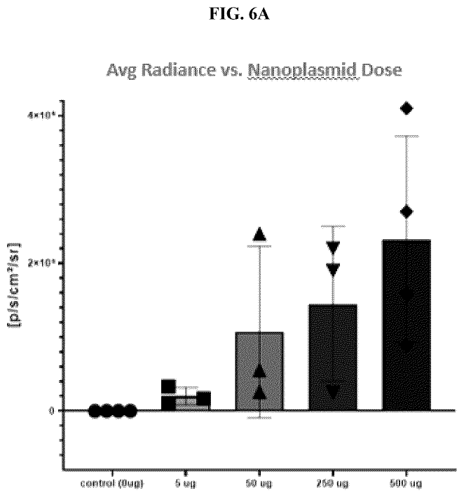

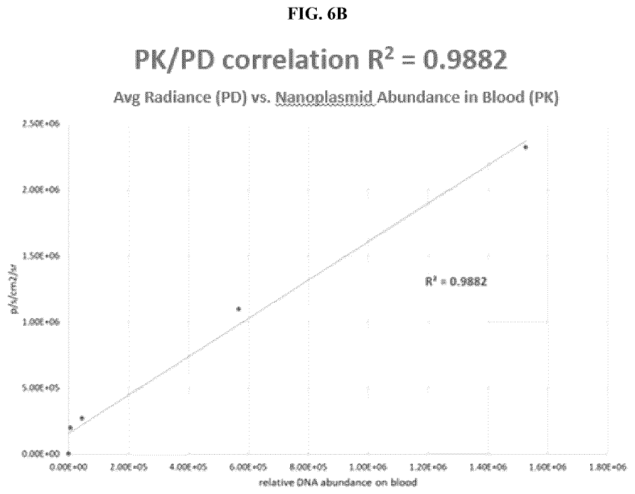

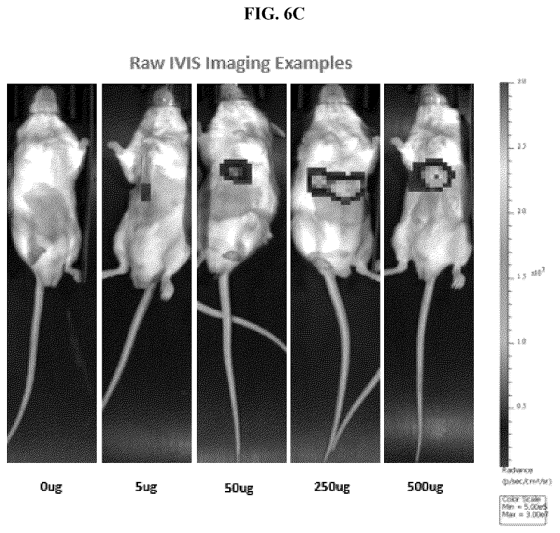

As shown in FIG. 6A, a greater average radiance is observed with increased nanoplasmid dose. Further, a linear relationship is observed between the average radiance and the abundance of the nanoplasmid in the blood (FIG. 6B). Raw IVIS images of mice are shown in FIG. 6C.

FIGS. 6A-6C depict results from IVIS of mouse liver after receiving 5 μg, 50 μg, 250 μg, or 500 μg of a luciferase nanoplasmid. FIG. 6A shows the average radiance (p/sec/cm²/sr) for each nanoplasmid dose tested. FIG. 6B shows the average radiance based on the relative DNA abundance in the blood. FIG. 6C shows an exemplary raw IVIS image for each nanoplasmid dose tested. Blue fluorescence is indicative of about 00.5*_{10\hat{ }7} p/sec/cm²/sr in fluorescence intensity; blue/green fluorescence is indicative of about 1*10\hat{ }7 p/sec/cm²/sr in fluorescence intensity; green fluorescence is indicative of about 1.5*_{10\hat{ }7} p/sec/cm²/sr in fluorescence intensity; green/yellow fluorescence is indicative of about 2*10\hat{ }7 p/sec/cm²/sr in fluorescence intensity; orange fluorescence is indicative of about 20.5*_{10\hat{ }7} p/sec/cm²/sr in fluorescence intensity; and red fluorescence is indicative of about 3*10\hat{ }7 p/sec/cm²/sr in fluorescence intensity. Increased fluorescence is indicative of a higher degree of luciferase expression. FIG. 6C at the left most panel shows no fluorescence for a 0 ug dose of nucleic acid construct administered; FIG. 6C at the left most panel shows no fluorescence for a 0 ug dose of nucleic acid construct administered; at the second from the left panel shows blue fluorescence for a 5 ug dose of nucleic acid construct administered; at the third from the left panel (middle panel) a larger area of blue/blue-green fluorescence for a 50 ug dose of nucleic acid construct administered; at the second from the right panel shows green fluorescence surrounded by blue fluorescence for a 250 ug dose of nucleic acid construct administered; and at the right panel shows green-yellow fluorescence surrounded by blue fluorescence, with observable red fluorescence in the center for a 500 ug dose of nucleic acid construct administered.

### Example 7: Kinetics and Durability of Transgene Expression in Mouse Liver

In this experiment, the kinetics of transgene expression were examined following sonoporation of a luciferase nanoplasmid into the mouse liver, as described in Example 3.

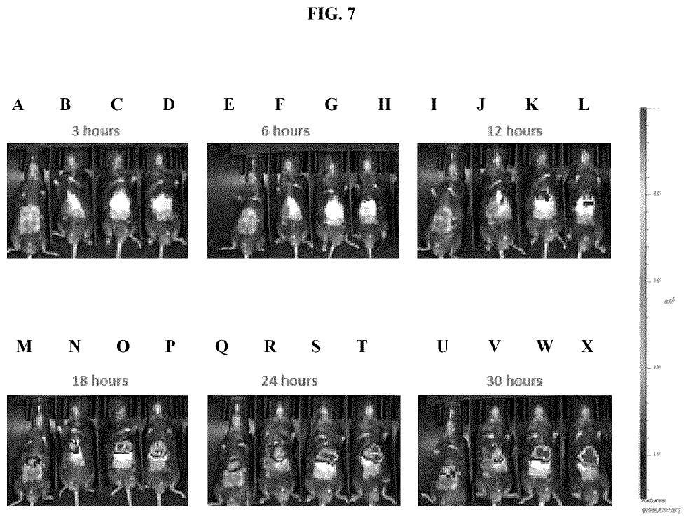

Transgene expression was assayed by IVIS 3, 6, 12, 18, 24, and 30 hours post-delivery. As shown in FIG. 7, transgene expression can first be detected 3 hours post-delivery, suggesting fast kinetics of DNA delivery to nuclei.

FIG. 7 depicts results from IVIS of mouse liver at 3, 6, 12, 18, 24, and 30 after delivery of a luciferase nanoplasmid by sonoporation in four different animals. Blue fluorescence is indicative of about 1*10\hat{ }5 p/sec/cm²/sr in fluorescence intensity; blue/green fluorescence is indicative of about 2*10\hat{ }5 p/sec/cm²/sr in fluorescence intensity; green fluorescence is indicative of about 3*10\hat{ }5 p/sec/cm²/sr in fluorescence intensity; yellow fluorescence is indicative of about 4*10\hat{ }5 p/sec/cm²/sr in fluorescence intensity; and red fluorescence is indicative of about 5*10\hat{ }5 p/sec/cm²/sr in fluorescence intensity. Increased fluorescence is indicative of a higher degree of luciferase expression. FIG. 7 shows no observable fluorescence at A-C, D-F, and U; shows blue fluorescence at D, H, and V-X; blue-green fluorescence at M, N, Q, and U; and yellow-red fluorescence a O, P, R-T, and V-X.

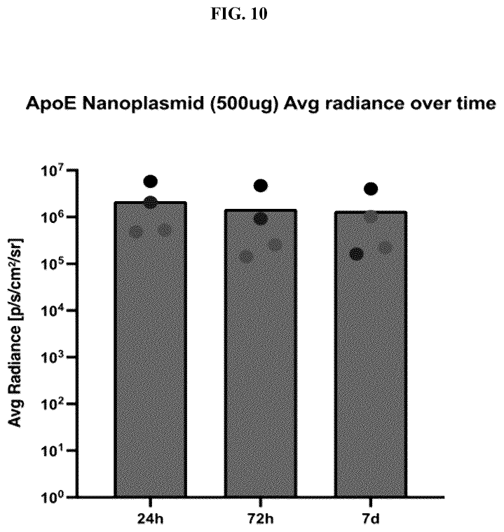

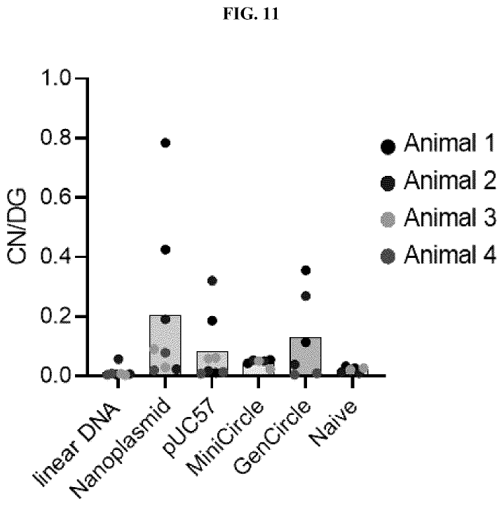

Further, it is observed that expression was maintained at consistent levels for 7 days, as shown by FIG. 10.

### Example 8: Safety Evaluations Following Treatment

In this experiment, various safety endpoints were evaluated after transgene delivery to the liver via sonoporation.

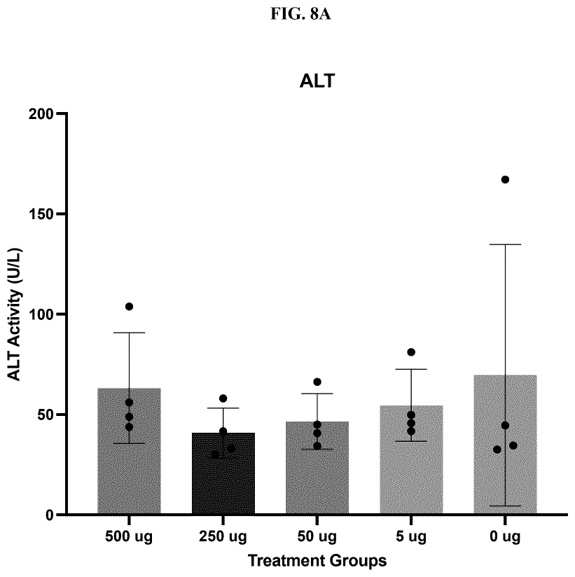

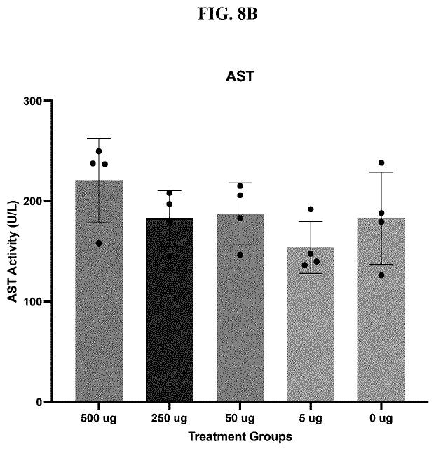

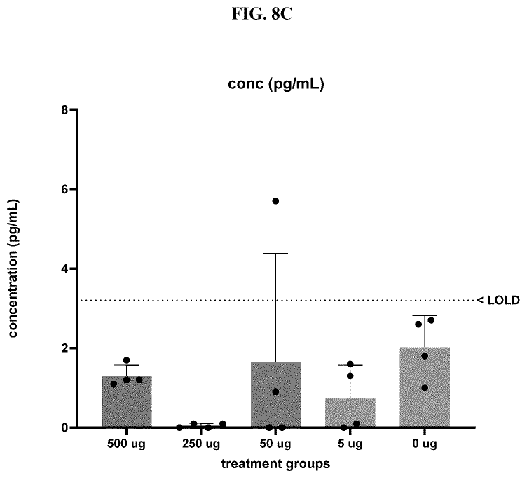

One day post-delivery, blood levels of ALT, AST, and IL6 were evaluated. As shown in FIGS. 8A-8C, no increase in ALT or AST activity was observed. Furthermore, the measured ALT and AST levels were within normal ranges based on previously published values. In BALB/C mice, normal ALT activity is between 40-170 U/L (female) or 41-131 (female) and normal AST activity is between 67-381 (female) or 55-381 (male). Similarly, no elevation in blood IL6 levels was observed (FIG. 8C).

FIG. 8A shows ALT activity (U/L) in the blood of mice transfected with the indicated nanoplasmid dose. FIG. 8B shows AST activity (U/L) in the blood of mice transfected with the indicated nanoplasmid dose. FIG. 8C shows the concentration of IL6 (pg/mL) in the blood of mice transfected with the indicated nanoplasmid dose.

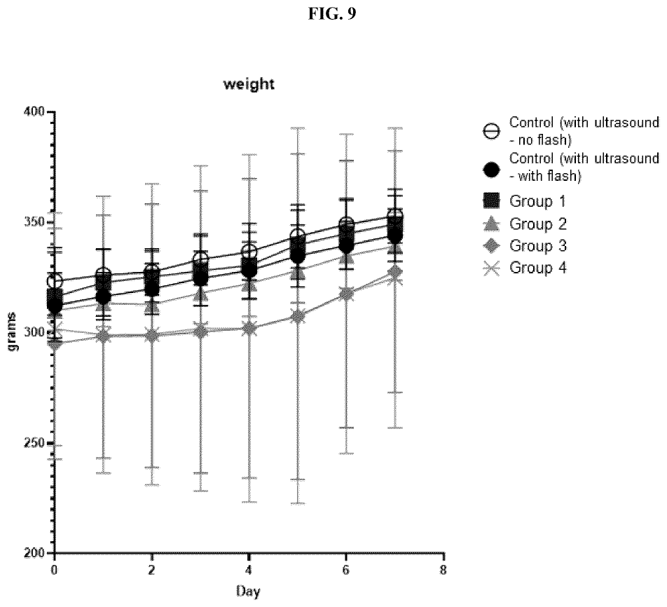

Body weight of treated animals was also examined for one week following delivery of a transgene under control of different promoters. As shown in FIG. 9, no decrease in body weight was observed, with the weight of treated animals following a similar trend as control animals.

Together, these data confirm safety of ultrasound-mediated transgene delivery to the liver.

### Example 9: Expression Levels of Exogenous DNA Delivered Via Sonoporation of Liver Cells Using Different Vectors

In this experiment, copy number per diploid genome of an exogenous gene in liver cells delivered via sonoporation using different expression vectors were measured using quantitative polymerase chain reaction (qPCR).

### Experimental Animals and Protocol

A vector encoding a firefly luciferase (Fluc) gene downstream of a CAG promoter was delivered by sonoporation to the livers of six groups of mice, each group comprising four Rag2 mice. Prior to sonoporation, 100 μg of DNA was administered to each mouse through a jugular vein catheter. A different vector was used in each group of mice to deliver the firefly luciferase (Fluc) gene. The vectors used in each group were as follows: Group 1, plasmid (pUC57-CAG-Fluc); Group 2, nanoplasmid (NTC9385R\(3×CpG)-CAG2.0 Fluc-CpG free BGH pA); Group 3, linear DNA (db312-001 TpUC CAG2.0-Fluc-CpG free bGHpA_pUC57); Group 4, GenCircle (GC-CAG-Fluc); group 5, MiniCircle (MC-CAG-Fluc); Group 6, negative control (no vector delivered).

One month after DNA delivery, the livers of the mice in all groups were harvested, and two liver samples per animal were analyzed. Genomic DNA (gDNA) was isolated from the liver samples using a QIAGEN AllPrep kit. Samples were handled and isolations were performed in a Mystaire Prep Station hood. DNA was diluted in TE buffer such that 50 ng of DNA was included in each qPCR reaction. qPCR reactions were run using TaqPath ProAmp MasterMix with Fluc5 p/ps.

Standard curves were generated using 1:100,000 through 1:1,000,000 serial dilutions of constructs in naïve gDNA from the six group samples as follows: (1) pUC57-CAG-Fluc (pUC57, 4.596 mg/ml, 6364 bp); (2) NTC9385R\(3×CpG)-CAG2.0 Fluc-CpG free BGH pA (Nanoplasmid, 4.93 mg/ml, 4092 bp); (3) db312-001 TpUC CAG2.0-Fluc-CpG free bGHpA_pUC57 (linear, 3.958 mg/ml, 4224 bp); (4) GC-CAG-Fluc (GenCircle, 5.0 mg/ml, 4083 bp); (5) MC-CAG-Fluc (MiniCircle, 5.0 mg/ml, 3699 bp); (6) untreated samples, copy number was calculated based on the average of parameters from the above standard curves

### Results

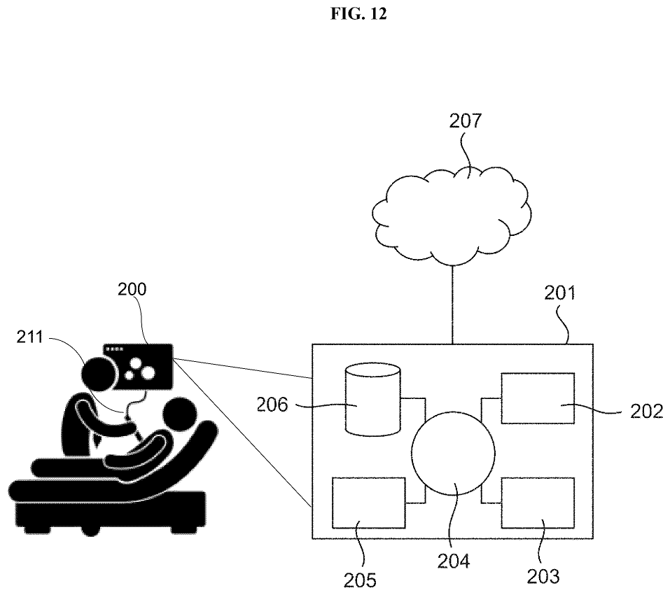

Using qPCR, Fluc transgene copy number per diploid genome in liver cells was measured. FIG. 12 provides mean transgene copy number of mice in the six groups in each of the two samples. Bar height indicates average transgene copy number in each group, and dots indicate sample measurement values for each animal. Fluc abundance was highest in group 2, in which the nanoplasmid vector was used. Using the sonoporation treatment protocol described herein, a significant increase in Fluc abundance as measured in copy number per diploid genome (CN/DG) was observed with the nanoplasmid vector, which was observed to outperform numerous other vectors including a standard plasmid (pUC57), a closed end linear DNA format, a modified plasmid DNA sequence with prokaryotic DNA sequences removed (minicircle), a modified small circular double stranded DNA vector having a vector backbone of about 430 bp with antibiotic resistance genes removed (GenCircle). In some cases, the measured Fluc abundance was in Group 2 was 2×-10× greater as compared to other vector formats.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of delivering a nucleic acid payload to a target cell of a subject comprising:
a. administering to the subject a nucleic acid construct comprising the nucleic acid payload, wherein the nucleic acid construct is a miniplasmid;
b. administering to the subject a plurality of sonoactive microstructures;
c. applying an ultrasonic acoustic energy to the target cell at a first mechanical index (MI) that is up to 0.4; and
d. applying an ultrasonic acoustic energy to the target cell at a second MI that is greater than 0.4 and up to 2.0.

2. The method of any one of the preceding claims, wherein an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject.

3. The method of any one of the preceding claims, wherein the miniplasmid is less than or equal to 500 base pairs in length excluding an expression cassette.

4. The method of any one of the preceding claims, wherein the nucleic acid construct is administered systemically.

5. The method of any one of the preceding claims, wherein applying the ultrasonic acoustic energy at the first MI and applying the ultrasonic acoustic energy at the second MI are repeated at least twice.

6. The method of any one of the preceding claims, wherein applying the ultrasonic acoustic energy at the first MI and applying the ultrasonic acoustic energy at the second MI are repeated from 4 to 18 times.

7. The method of any one of the preceding claims, wherein applying the ultrasonic acoustic energy at the first MI and applying the ultrasonic acoustic energy at the second MI are repeated from 6 to 12 times.

8. The method of any one of the preceding claims, wherein applying the ultrasonic acoustic energy at the first MI and applying the ultrasonic acoustic energy at the second MI are repeated from 8 to 10 times.

9. The method of any one of the preceding claims, wherein applying the ultrasonic acoustic energy at the first MI and applying the ultrasonic acoustic energy at the second MI are repeated 9 times.

10. The method of any one of the preceding claims, wherein an ultrasound transducer is continuously in contact with the subject during applying the ultrasonic acoustic energy at the first MI and applying the ultrasonic acoustic energy at the second MI.

11. The method of any one of the preceding claims, wherein an ultrasound transducer sends ultrasound acoustic energy or receives reflected ultrasound acoustic energy at least 95% of a period of time in which an ultrasound transducer continuously is contacting the subject.

12. The method of any one of the preceding claims, wherein applying the ultrasonic acoustic energy at the second MI comprises applying the ultrasonic acoustic energy at the second MI using a pulse.

13. The method of any one of the preceding claims, wherein applying the ultrasonic acoustic energy at the second MI comprises applying the ultrasonic acoustic energy at the second MI using a pulse with a duration of about 1 μs to about 500 μs.

14. The method of any one of the preceding claims, wherein applying the ultrasonic acoustic energy at the second MI comprises applying the ultrasonic acoustic energy at the second MI using a pulse with a duration of about 100 μs to about 3300 μs.

15. The method of any one of the preceding claims, wherein applying the ultrasonic acoustic energy at the second MI comprises applying the ultrasonic acoustic energy at the second MI using a pulse with a duration of about 200 μs.

16. The method of any one of the preceding claims, wherein applying the ultrasonic acoustic energy at the second MI comprises applying the ultrasonic acoustic energy at the second MI using a pulse with a duration of up to 200 μs.

17. The method of any one of the preceding claims, wherein applying the ultrasonic acoustic energy at the second MI comprises applying the ultrasonic acoustic energy at the second MI using a pulse with a duration of up to 500 μs.

18. The method of any one of the preceding claims, wherein applying the ultrasonic acoustic energy at the second MI comprises applying the ultrasonic acoustic energy at the second MI using a pulse with a duration of about 1 μs to about 200 μs.

19. The method of any one of the preceding claims, applying the ultrasonic acoustic energy at the second MI comprises applying the ultrasonic acoustic energy at the second MI using a pulse with a duration of about 1 μs to about 5 μs.

20. The method of any one of the preceding claims, wherein applying the ultrasonic acoustic energy at the second MI comprises applying the ultrasonic acoustic energy at the second MI using a pulse with a duration of about 2.3 μs.

21. The method of any one of the preceding claims, wherein applying the ultrasonic acoustic energy at the second MI comprises applying the ultrasonic acoustic energy at the second MI using a pulse with a duration of at least 2.3 μs.

22. The method of any one of the preceding claims, wherein applying the ultrasonic acoustic energy at the first MI comprises initially applying the ultrasonic acoustic energy at the first MI from about 2 s to about 30 s.

23. The method of any one of the preceding claims, further comprising repeating the applying the ultrasonic acoustic energy at the first MI, and the applying the ultrasonic acoustic energy at the second MI.

24. The method of claim 23 wherein the repeating comprises applying the ultrasonic acoustic energy at the first MI for an amount of time sufficient to permit reperfusion of the sonoactive microstructures in a tissue comprising the target cell.

25. The method of claim 23, wherein the repeating comprises applying the ultrasonic acoustic energy at the first MI for 1-30 seconds before repeating the applying the ultrasound acoustic energy at the second MI.

26. The method of claim 23, wherein the repeating comprises applying the ultrasonic acoustic energy at the first MI for 5-15 seconds before applying the ultrasound acoustic energy at the second MI.

27. The method of any one of the preceding claims, wherein the repeating comprises applying the ultrasonic acoustic energy at the first MI for 10 seconds before applying the ultrasound acoustic energy at the second MI.

28. The method of any one of the preceding claims, wherein the ultrasonic acoustic energy of (c) and the ultrasonic acoustic energy of (d) are applied for a total amount of time ranging from about 1 s to about 60 m.

29. The method of any one of the preceding claims, wherein the ultrasonic acoustic energy of (c) and the ultrasonic acoustic energy of (d) are applied for a total amount of time ranging from about 60 s to about 120 s.

30. The method of any one of the preceding claims, wherein the first MI ranges from about 0.05 to about 0.3.

31. The method of any one of the preceding claims, wherein the first MI ranges from about 0.09 to about 0.3.

32. The method of any one of the preceding claims, wherein the second MI ranges from about 1.0 to about 1.8.

33. The method of any one of the preceding claims, wherein the second MI ranges from about 1.4 to about 1.8.

34. The method of any one of the preceding claims, wherein the second MI ranges from about 1.4 to about 2.0.

35. The method of any one of the preceding claims, wherein the miniplasmid does not comprise antibiotic resistant genes.

36. The method of any one of the preceding claims, wherein the miniplasmid does not comprise a bacterial genome.

37. The method of any one of the preceding claims, wherein the nucleic acid construct enhances the expression of a nonendogenous gene within the miniplasmid.

38. The method of any one of the preceding claims, wherein the method induces expression of the nucleic acid payload in the target cell within 20 hours of the applying the ultrasonic acoustic energy.

39. The method of any one of the preceding claims, wherein the nucleic acid construct is configured to perform gene augmentation, gene replacement, base editing, base knockdown, gene editing gene knockdown, or gene knockout.

40. The method of any one of the preceding claims, wherein the nucleic acid construct is configured for enhanced stability in vivo.

41. The method of any one of the preceding claims, wherein the nucleic acid construct is administered at a dose of about 100 ug to about 200 ug.

42. The method of any one of the preceding claims, wherein the nucleic acid construct is administered at a dose of about 0.5 mg/kg to about 32 mg/kg.

43. The method of any one of the preceding claims, wherein about 2×10\hat{ }13 to about 3×10\hat{ }13 copies of the nucleic acid construct are administered to the subject.

44. The method of any one of the preceding claims, wherein the miniplasmid comprises a therapeutic transgene and/or a regulatory element.

45. The method of any one of the preceding claims, wherein the sonoactive microstructures are microbubbles.

46. The method of any one of the preceding claims, wherein applying ultrasonic acoustic energy at the first MI induces stable vibrational cavitation of the sonoactive microstructures.

47. The method of any one of the preceding claims, wherein applying ultrasonic acoustic energy at the first MI does not induce substantial disruption (e.g., bursting or inertial cavitation) of the sonoactive microstructures.

48. The method of any one of the preceding claims, wherein applying ultrasonic acoustic energy at the first MI does not induce substantial disruption of the sonoactive microstructures in a vasculature space and an extravascular space, or induces stable vibration cavitation of the sonoactive microstructures in a vasculature space and an extravascular space.

49. The method of any one of the preceding claims, wherein applying ultrasonic acoustic energy at the second MI induces inertial cavitation of the sonoactive microstructures to disrupt the sonoactive microstructures.

50. The method of any one of the preceding claims, wherein applying ultrasonic acoustic energy at the second MI induces inertial cavitation of the sonoactive microstructures to disrupt the sonoactive microstructures in a vascular space and an extravascular space.

51. The method of any one of the preceding claims, wherein the extravascular spaces comprise an interstitial space, a subcutaneous space, an intramuscular inter-osseous space, or a lymphatic space.

52. The method of any one of the preceding claims, wherein the extravascular spaces comprise an extravascular tissue.

53. The method of any one of the preceding claims, wherein the extravascular tissue comprises an interstitial space, a cytoplasmic space, a subcutaneous, a lymph tissues, a muscle, or combinations thereof.

54. The method of any one of the preceding claims, wherein the method does not result in substantial cellular damage to the target cell.

55. The method of any one of the preceding claims, wherein the method results in less than 1%, 5%, or 10% of target cells undergoing apoptosis.

56. The method of any one of the preceding claims, wherein the following biomarkers for cellular damage are not detected at apoptotic levels following delivering the nucleic acid payload to the target cell of the subject: ALT, AST, IL6, BCL2, or combinations thereof, and, optionally wherein the target cell is in a liver.

57. The method of any one of the preceding claims, wherein the following biomarkers for cellular damage are not clinically elevated following delivering the nucleic acid payload to the target cell of the subject: ALT, AST, IL6, BCL2, or combinations thereof, and, optionally wherein the target cell is in a liver.

58. The method of any one of the preceding claims, wherein ALT is not detected at levels exceeding 60, 65, 70, 75, 80, 85, 90, 95, 100, 125, 150, 175, or 200 U/L following delivery of the nucleic acid payload to the target cell of the subject.

59. The method of any one of the preceding claims, wherein AST is not detected at levels exceeding 225, 250, 275, or 300 U/L following delivery of the nucleic acid payload to the target cell of the subject.

60. The method of any one of the preceding claims, wherein IL6 is not detected at levels exceeding 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, or 6 pg/mL following delivery of the nucleic acid payload to the target cell of the subject.

61. The method of any one of the preceding claims, wherein the target cell is in a liver.

62. The method of any one of the preceding claims, wherein the target cell is in a kidney.

63. The method of any one of the preceding claims, wherein the target cell is in a heart or skeletal muscle.

64. The method of any one of the preceding claims, wherein the target cell is in a brain.

65. The method of any one of the preceding claims, wherein the target cell is in a pancreas.

66. The method of any one of the preceding claims, wherein the target cell is in a tumor, or is a tumor cell.

67. The method of any one of the preceding claims, wherein applying the ultrasound acoustic energy at the first mechanical index induces formation of an intercellular gap or an interendothelial gap.

68. The method of any one of the preceding claims, wherein the intercellular gap or the interendothelial gap ranges from about 10 nm to about 10 um.

69. The method of any one of the preceding claims, further comprising moving the nucleic acid construct from an intravenous space into an interstitial space.

70. The method of any one of the preceding claims, further comprising moving the nucleic acid construct from an interstitial space to an intracellular space.

71. The method of any one of the preceding claims, wherein the stable vibration cavitation of the sonoactive microstructures moves the nucleic acid construct from an intravenous space into an interstitial space.

72. The method of any one of the preceding claims, wherein the inertial cavitation further moves the nucleic acid construct from an interstitial space into an intracellular space.

73. The method of any one of the preceding claims, wherein applying the ultrasound acoustic energy at the second mechanical index induces formation of a pore in a membrane of the cell.

74. The method of any one of the preceding claims, wherein the formation of a pore in a membrane of the cell ranges from about 10 nm to about 10 um.

75. The method of any one of the preceding claims, wherein the nucleic acid payload comprises a transgene.

76. The method of any one of the preceding claims, wherein the transgene comprises a therapeutic transgene.

77. The method of any one of the preceding claims, wherein the transgene comprises a detectible marker.

78. The method of any one of the preceding claims, wherein the transgene comprises luciferase.

79. The method of any one of the preceding claims, wherein the nucleic acid construct comprises a promoter sequence comprising CAG.

80. The method of any one of the preceding claims, wherein the nucleic acid construct comprises a promoter sequence comprising ApoE.

81. The method of any one of the preceding claims, wherein the nucleic acid construct comprises a promoter sequence comprising SERP.

82. The method of any one of the preceding claims, wherein the nucleic acid construct comprises a promoter sequence comprising P3.

83. The method of any one of the preceding claims, further comprising inducing expression of the nucleic acid payload in the target cell.

84. The method of claim 83, wherein the inducing expression of the nucleic acid payload comprises inducing expression of luciferase.

85. The method of claim 83, wherein inducing expression of the nucleic acid payload comprises inducing a flux of at least 10\hat{ }6 p/s.

86. The method of claim 83, wherein inducing expression of the nucleic acid payload comprises inducing a flux of about 10\hat{ }6 p/s to about 10\hat{ }9 p/s.

87. The method of claim 83, wherein inducing expression of the nucleic acid payload comprises inducing a flux which is 2, 3, 4, or 5× greater than expression induced without repeating applying the ultrasonic acoustic energy at the first MI, and the applying the ultrasonic acoustic energy at the second MI.

88. The method of claim 83, wherein inducing expression of the nucleic acid payload comprises inducing production of RNA encoded by the payload.

89. The method of claim 83, wherein inducing expression of the nucleic acid payload comprises inducing production of protein encoded by the payload.

90. The method of any one of the preceding claims, wherein the sonoactive microstructures are administered at a concentration of about 5×10\hat{ }8 to about 1.2×10\hat{ }9 microstructures/mL.

91. The method of any one of the preceding claims, wherein the sonoactive microstructures comprise a lipid-stabilized microstructure.

92. The method of any one of the preceding claims, wherein the sonoactive microstructures comprise a phospholipid-stabilized microstructure.

93. The method of claim 92, wherein the sonoactive microstructures comprise Sonazoid microbubbles.

94. The method of any one of the preceding claims, wherein the phospholipid-stabilized microstructure comprises a high molecular weight gas core, or a perflutran core.

95. The method of claim 94, wherein the sonoactive microstructures are administered at a concentration of about 10\hat{ }9 microstructures/mL.

96. The method of any one of the preceding claims, wherein the sonoactive microstructures are administered at a concentration of about 0.1 to about 20.0 mL/kg.

97. The method of any one of the preceding claims, wherein the sonoactive microstructures are administered at a concentration of about 0.1 to about 0.8 mg/kg.

98. The method of any one of the preceding claims, wherein the sonoactive microstructures comprise a protein stabilized microstructure.

99. The method of claim 98, wherein the sonoactive microstructures comprise optison microbubbles.

100. The method of claim 98, wherein the sonoactive microstructures are administered at a concentration of about 5×10\hat{ }8 to about 8×10\hat{ }8 microstructures/mL.

101. The method of any one of the preceding claims, wherein the ultrasound acoustic energy is applied at a distance of about 0.5 cm to about 20 cm from the target cell.

102. The method of any one of the preceding claims, wherein the nucleic acid construct and the sonoactive microstructures are coadministered.

103. The method of claim 102, wherein the nucleic acid construct and the sonoactive microstructures are mixed prior to being coadministered.

104. The method of any one of the preceding claims, wherein the administering of the nucleic acid construct and the sonoactive microstructures occurs serially, concurrently, sequentially, or continuously.

105. The method of any one of the preceding claims, wherein the administering of the nucleic acid construct and the sonoactive microstructures occurs serially.

106. The method of any one of the preceding claims, wherein the administering of the nucleic acid construct and the sonoactive microstructures occurs concurrently.

107. The method of any one of the preceding claims, wherein the administering of the nucleic acid construct and the sonoactive microstructures occurs sequentially.

108. The method of any one of the preceding claims, wherein the administering of the nucleic acid construct and the sonoactive microstructures occurs continuously.

109. The method of any one of the preceding claims, wherein the administering of the nucleic acid construct and the sonoactive microstructures is by intravenous administration.

110. The method of any one of the preceding claims, wherein the administering of the nucleic acid construct and the sonoactive microstructures intramuscular, subcutaneous, inter-osseous or retrovesiclar administration.

111. The method of any one of the preceding claims, wherein inducing expression of the nucleic acid payload comprises inducing expression within about 3 hours of administering the payload.

112. The method of any one of the preceding claims, wherein inducing expression of the nucleic acid payload comprises inducing expression within about 6 hours of administering the payload.

113. The method of any one of the preceding claims, wherein inducing expression of the nucleic acid payload comprises inducing expression within about 12 hours of administering the payload.

114. The method of any one of the preceding claims, wherein inducing expression of the nucleic acid payload comprises inducing expression in a cell in a liver.

115. The method of any one of the preceding claims, wherein inducing expression of the nucleic acid payload comprises inducing expression in a cell in a kidney.

116. The method of any one of the preceding claims, further comprising inducing expression of the nucleic acid payload and maintaining expression of a protein encoded by the nucleic acid payload for at least 1, 2, 3, 4, 5, 6, or 7 days.

117. The method of any one of the preceding claims, further comprising inducing expression of the nucleic acid payload and maintaining expression of a protein encoded by the nucleic acid payload for at least 1, 2, 3, 4, 5, 6, or 7 days.

118. The method of any one of the preceding claims, wherein the method increases durability of expression of a protein encoded by the nucleic acid payload.

119. The method of any one of the preceding claims, further comprising increasing expression of the nucleic acid payload by increasing the dosage of the nucleic acid payload administered to the subject.

120. The method of any one of the preceding claims, further comprising increasing expression of the nucleic acid payload by increasing the dosage of the nucleic acid payload administered to the subject in a linear manner.

121. The method of any one of the preceding claims, further comprising increasing expression of the nucleic acid payload by administering at least 5, 50, 250, or 500 μg of the nucleic acid payload to the subject.

122. The method of any one of the preceding claims, wherein delivering the nucleic acid payload to the target cell of the subject increases or decreases expression of a gene in the target cell.

123. The method of any one of the preceding claims, wherein the nucleic acid payload is delivered to the target cell, resulting in a copy number of the nucleic acid payload per diploid genome of at least 0.15.

124. The method of any one of the preceding claims, wherein the nucleic acid payload is delivered to the target cell, resulting in a copy number of the nucleic acid payload per diploid genome of at least 0.2.

125. The method of any one of the preceding claims, wherein the nucleic acid payload is delivered to the target cell, resulting in a copy number of the nucleic acid payload per diploid genome of 0.15 to 0.3.

126. A kit comprising:
a. a first container comprising microbubbles for sonoporation; and
b. a second container comprising miniplasmids comprising a transgene.

127. The kit of claim 126, wherein the miniplasmid further comprises an expression cassette.

128. The kit of claim 126, wherein the first container and second container are configured to induce the expression of the transgene in the target cell of the subject within 20 hours after the transfection.

129. The kit of claim 126, further comprising instructions for operation of an ultrasound machine hardware and software parameters sufficient to disrupt the sonoactive microstructures.

130. The kit of claim 126, further comprising instructions for administration of the first container and the second container.

131. A system comprising:
an ultrasound transducer configured to apply ultrasound acoustic energy to a subject at a plurality of mechanical indexes;
a computer system comprising a computer processor and a computer-readable medium, wherein the computer system is configured to implement a method of applying ultrasonic acoustic energy to a target cell of the subject, the method comprising:
a. applying an ultrasonic acoustic energy to the target cell at a first mechanical index (MI) that is up to 0.4; and
b. applying an ultrasonic acoustic energy to the target cell at a second MI that is greater than 0.4 and up to 2.0,
wherein the subject has been administered (1) a nucleic acid construct comprising a nucleic acid payload, wherein the nucleic acid construct is a miniplasmid, and (2) a plurality of sonoactive microstructures.

132. The system of claim 131, wherein the ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject.

133. The system of claim 131, wherein the miniplasmid is less than or equal to 500 base pairs in length excluding an expression cassette

134. The system of claim 131, wherein applying the ultrasonic acoustic energy at the first MI and applying the ultrasonic acoustic energy at the second MI are repeated at least twice.

135. The system of claim 131, wherein applying the ultrasonic acoustic energy at the first MI and applying the ultrasonic acoustic energy at the second MI are repeated from 4 to 18 times.

136. The system of claim 131, wherein applying the ultrasonic acoustic energy at the first MI and applying the ultrasonic acoustic energy at the second MI are repeated from 6 to 12 times.

137. The system of claim 131, wherein applying the ultrasonic acoustic energy at the first MI and applying the ultrasonic acoustic energy at the second MI are repeated from 8 to 10 times.

138. The system of claim 131, wherein the second MI ranges from about 1.4 to about 2.0.

139. The system of claim 131, wherein applying the ultrasound acoustic energy at the second mechanical index induces formation of a pore in a membrane of the cell.

140. The system of claim 131, wherein applying the ultrasound acoustic energy at the first mechanical index induces formation of an intercellular gap or an interendothelial gap.

141. The system of claim 131, wherein an ultrasound transducer sends ultrasound acoustic energy or receives reflected ultrasound acoustic energy at least 95% of a period of time in which an ultrasound transducer continuously is contacting the subject.

142. The system of claim 131, wherein applying the ultrasonic acoustic energy at the second MI comprises applying the ultrasonic acoustic energy at the second MI using a pulse.

143. The system of claim 131, wherein applying the ultrasonic acoustic energy at the second MI comprises applying the ultrasonic acoustic energy at the second MI using a pulse with a duration of about 1 μs to about 500 μs.

144. The system of claim 131, wherein applying the ultrasonic acoustic energy at the second MI comprises applying the ultrasonic acoustic energy at the second MI using a pulse with a duration of up to 200 μs.

145. The system of claim 131, wherein applying the ultrasonic acoustic energy at the second MI comprises applying the ultrasonic acoustic energy at the second MI using a pulse with a duration of up to 500 μs.

146. The system of claim 131, wherein applying the ultrasonic acoustic energy at the second MI comprises applying the ultrasonic acoustic energy at the second MI using a pulse with a duration of about 1 μs to about 200 μs.

147. The system of claim 131, wherein applying the ultrasonic acoustic energy at the second MI comprises applying the ultrasonic acoustic energy at the second MI using a pulse with a duration of about 2.3 μs.

148. The system of claim 131, further comprising repeating the applying the ultrasonic acoustic energy at the first MI, and the applying the ultrasonic acoustic energy at the second MI.

149. The system of claim 148, wherein the repeating comprises applying the ultrasonic acoustic energy at the first MI for an amount of time sufficient to permit reperfusion of the sonoactive microstructures in a tissue comprising the target cell.

150. The system of claim 148, wherein the repeating comprises applying the ultrasonic acoustic energy at the first MI for 1-30 seconds before repeating the applying the ultrasound acoustic energy at the second MI.

151. The system of claim 148, wherein the repeating comprises applying the ultrasonic acoustic energy at the first MI for 5-15 seconds before applying the ultrasound acoustic energy at the second MI.

152. The system of claim 148, wherein the repeating comprises applying the ultrasonic acoustic energy at the first MI for 10 seconds before applying the ultrasound acoustic energy at the second MI.

153. A computer-readable medium configured to implement a method of applying ultrasonic acoustic energy to a target cell of the subject, the method comprising:
a. applying an ultrasonic acoustic energy to the target cell at a first mechanical index (MI) that is up to 0.4; and
b. applying an ultrasonic acoustic energy to the target cell at a second MI that is greater than 0.4 and up to 2.0,
wherein the subject has been administered (1) a nucleic acid construct comprising a nucleic acid payload, wherein the nucleic acid construct is a miniplasmid, and (2) a plurality of sonoactive microstructures.

154. The computer readable medium of claim 153, wherein an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject.

155. The computer readable medium of claim 153, wherein the miniplasmid is less than or equal to 500 base pairs in length excluding an expression cassette.

156. The computer readable medium of claim 153, wherein applying the ultrasonic acoustic energy at the first MI and applying the ultrasonic acoustic energy at the second MI are repeated at least twice.

157. The computer readable medium of claim 153, wherein applying the ultrasonic acoustic energy at the first MI and applying the ultrasonic acoustic energy at the second MI are repeated from 4 to 18 times.

158. The computer readable medium of claim 131, wherein applying the ultrasonic acoustic energy at the first MI and applying the ultrasonic acoustic energy at the second MI are repeated from 6 to 12 times.

159. The computer readable medium of claim 153, wherein applying the ultrasonic acoustic energy at the first MI and applying the ultrasonic acoustic energy at the second MI are repeated from 8 to 10 times.

160. The computer readable medium of claim 153, wherein the second MI ranges from about 1.4 to about 2.0.

161. The computer readable medium of claim 153, wherein applying the ultrasound acoustic energy at the second mechanical index induces formation of a pore in a membrane of the cell.

162. The computer readable medium of claim 153, wherein applying the ultrasound acoustic energy at the first mechanical index induces formation of an intercellular gap or an interendothelial gap.

163. The computer readable medium of claim 153, wherein an ultrasound transducer sends ultrasound acoustic energy or receives reflected ultrasound acoustic energy at least 95% of a period of time in which an ultrasound transducer continuously is contacting the subject.

164. The computer readable medium of claim 153, wherein applying the ultrasonic acoustic energy at the second MI comprises applying the ultrasonic acoustic energy at the second MI using a pulse.

165. The computer readable medium of claim 153, wherein applying the ultrasonic acoustic energy at the second MI comprises applying the ultrasonic acoustic energy at the second MI using a pulse with a duration of about 1 μs to about 500 μs.

166. The computer readable medium of claim 153, wherein applying the ultrasonic acoustic energy at the second MI comprises applying the ultrasonic acoustic energy at the second MI using a pulse with a duration of up to 200 μs.

167. The computer readable medium of claim 153, wherein applying the ultrasonic acoustic energy at the second MI comprises applying the ultrasonic acoustic energy at the second MI using a pulse with a duration of up to 500 μs.

168. The computer readable medium of claim 153, wherein applying the ultrasonic acoustic energy at the second MI comprises applying the ultrasonic acoustic energy at the second MI using a pulse with a duration of about 1 μs to about 200 μs.

169. The computer readable medium of claim 153, wherein applying the ultrasonic acoustic energy at the second MI comprises applying the ultrasonic acoustic energy at the second MI using a pulse with a duration of about 2.3 μs.

170. The computer readable medium of claim 153, further comprising repeating the applying the ultrasonic acoustic energy at the first MI, and the applying the ultrasonic acoustic energy at the second MI.

171. The computer readable medium of claim 170, wherein the repeating comprises applying the ultrasonic acoustic energy at the first MI for an amount of time sufficient to permit reperfusion of the sonoactive microstructures in a tissue comprising the target cell.

172. The computer readable medium of claim 170, wherein the repeating comprises applying the ultrasonic acoustic energy at the first MI for 1-30 seconds before repeating the applying the ultrasound acoustic energy at the second MI.

173. The computer readable medium of claim 170, wherein the repeating comprises applying the ultrasonic acoustic energy at the first MI for 5-15 seconds before applying the ultrasound acoustic energy at the second MI.

174. The computer readable medium of claim 170, wherein the repeating comprises applying the ultrasonic acoustic energy at the first MI for 10 seconds before applying the ultrasound acoustic energy at the second MI.
